(19) United States
(12) Patent Application Publication
(10) Pub. No.: US 2016/0282432 A1
(43) Pub. Date: Sep. 29, 2016

(54) Omni-Tomographic Imaging for Interior Reconstruction using Simultaneous Data Acquisition from Multiple Imaging Modalities

(71) Applicant: Ge Wang, Latham, NY (US)

(72) Inventor: Ge Wang, Latham, NY (US)

(73) Assignee: Virginia Tech Intellectual Properties, Inc., Blacksburg, VA (US)

(21) Appl. No.: 15/064,932

(22) Filed: Mar. 9, 2016

Related U.S. Application Data
(63) Continuation of application No. 13/439,419, filed on Apr. 4, 2012, now abandoned.
(60) Provisional application No. 61/471,245, filed on Apr. 4, 2011.
(60) Provisional application No. 61/495,422, filed on Jun. 10, 2011.

(51) Int. Cl. G01R 33/48; A61B 5/00; A61B 6/00; A61B 5/055

(52) U.S. Cl. CPC G01R 33/4812; A61B 5/055; A61B 5/704; A61B 5/0035; A61B 5/0059; A61B 6/485; G01R 33/481; A61B 6/032

(57) ABSTRACT

Embodiments of the invention relate to omni-tomographic imaging or grand fusion imaging, i.e., large scale fusion of simultaneous data acquisition from multiple imaging modalities such as CT, MRI, PET, SPECT, US, and optical imaging. A preferred omni-tomography system of the invention comprises two or more imaging modalities operably configured for concurrent signal acquisition for performing ROI-targeted reconstruction and contained in a single gantry with a first inner ring as a permanent magnet; a second middle ring containing an x-ray tube, detector array, and a pair of SPECT detectors; and a third outer ring for containing PET crystals and electronics. Omni-tomography offers great synergy in vivo for diagnosis, intervention, and drug development, and can be made versatile and cost-effective, and as such is expected to become an unprecedented imaging platform for development of systems biology and modern medicine.

### CROSS-REFERENCE TO RELATED APPLICATIONS

The present application claims priority to and the benefit of the filing date of U.S. Provisional Application No. 61/471,245, filed Apr. 4, 2011, and 61/495,422, filed Jun. 10, 2011, the disclosures of which are hereby incorporated by reference herein in their entireties.

### STATEMENT OF GOVERNMENT INTEREST

This invention was made with government support under Contract No. EB011785 awarded by NIH/NIBIB and Contract No. HL098912 awarded by NIH/NHLBI. The government has certain rights in the invention.

### BACKGROUND OF THE INVENTION

1. Field of the Invention

The present invention relates to the field of medical imaging. More particularly, embodiments of the invention relate to methods, systems, and devices for omni-tomographic imaging or grand fusion imaging, i.e., large scale fusion of simultaneous data acquisition from multiple imaging modalities such as CT, MRI, PET, SPECT, US, and optical imaging. Included in embodiments is an omni-tomographic scanner using interior MRI with a non-homogeneous magnetic field, interior CT, interior SPECT, and interior x-ray fluorescence tomography, all of which are in the compressive sensing framework.

2. Description of Related Art

Tomography is widely used for preclinical and clinical imaging to characterize morphology, and to a limited extent biological function (e.g., physiology). Given current technology, several intrinsic limitations of tomography exist, especially the necessity to acquire, reconstruct and analyze data obtained sequentially on the same subject with or without explicit superimposition of the results. This separation in time impairs the ability to decipher and understand biological functions, as they are certainly dynamic processes (especially relative to morphological changes). For example, a cardiac infarct commonly begins with decreased perfusion, then tissue hypoxia and eventually cell death; these stages exist in a continuum that can evolve over a short time frame relative to the time needed to acquire multimodality imaging data.

If technology could be developed to simultaneously image physiome and other dynamic complexities with multiple modalities, biological processes which evolve rapidly may become transparent. Such processes have temporal evolution at many intervals, including mille- or micro-seconds, seconds, minutes, days or longer, and may or may not be reversible. A single session imaging time frame is important to image processes such as: ischemia, drug interactions, radiation effects, apoptosis, and many others. To some extent, this has already been accomplished with PET-CT and MRI-PET systems. Although the delay in data acquisition is improved relative to single-modality predecessors, many of these multimodality imaging systems still acquire data sequentially instead of simultaneously.

Now that a formal mechanism for unifying structural and functional data is attracting interest, especially with the Physiome project, the need to simultaneously acquire and unify multimodal images has become more important than ever before. There are critical and immediate needs to remove the limitations inherent in today's tomographic imaging approaches to the extent that complex dynamic biological processes can be studied in vivo and in real time using multiple modalities.

There a numerous modalities existing today that can be used in multi-modality configurations. Projection x-ray imaging revolutionized medical diagnostics and inspired the development of other tomographic imaging modalities. As a result, an impressive array of scanners now exist for computed tomography (CT), magnetic resonance imaging (MRI), positron emission tomography (PET) and single photon emission computed tomography (SPECT), ultrasonic imaging (US) Imaging, optical imaging, and more. These modalities utilize different physical principles and reveal information from distinct but complementary perspectives. A recent book chapter on the state-of-the-art in medical imaging, from several of the inventors of this application, provides further details and references. See Wang G, G. H., Zhang J. Weir V, Xu X C. Cong A, Bennett J, Gao H, Medical Imaging, in Handbook of Research on Biomedical Engineering Education and Advanced Bioengineering Learning: Interdisciplinary Concepts (two-volume set). Z. O. Abu-Faraj, Editor. In press, IGI Global: Hershey, Pa.

Another modality, X-ray CT (otherwise referred to as CT), has been rather popular over the past two decades: about 100 million scans are performed annually in the United States alone. With inherently higher spatial resolution (about 0.3 mm) and faster imaging speeds (about 100 ms) (see Petersilka, M., et al., Technical principles of dual source CT. European Journal of Radiology, 2008. 68(3): p. 362-368), CT can examine tissues that differ in density by less than 1%, along with physiological and pathological dynamics. A single circular CT scan can cover many important biomedical targets with its large longitudinal range (up to 16 cm).

Moreover, a spiral cone-beam CT scan further extends this coverage to solve a long object problem (e.g., whole body angiography). In particular. U.S. Pat. No. 8,121,249 entitled “Multi-Parameter X-Ray Computed Tomography,” describes methods for extracting x-ray small-angle scattering data and using this segregated data to produce a high quality, high contrast x-ray image, including collecting x-ray projection data multiple times with varying collimation before an x-ray detector array using different collimation aspect ratios. The projection data acquired with a collimator of a sufficiently large aspect ratio (otherwise referred to as a high collimation aspect ratio) contain mainly the primary beam with little scattering. In contrast, the corresponding data acquired with an appropriately reduced collimation aspect ratio (otherwise referred to as a small or low collimation aspect ratio) include both small-angle scattering signals and the primary beam signals. Analysis of these paired or corresponding datasets (e.g., by digital subtraction of one dataset from the other) will produce or isolate the desired dark-field signals, in addition to traditional transmission measurement. The primary shortcomings of current CT scanners, however; are patient radiation dose and limited contrast resolution, both of which can be significantly improved using contrast agents.

Other imaging techniques include those disclosed in U.S. patent application Ser. No. 12/914,790, filed Oct. 28, 2010, and entitled “Cardiac Computed Tomography Methods and Systems Using Fast Exact/Quasi-Exact Filtered Back Projection Algorithms,” which provide methods and systems for reconstructing an image from projection data provided by a computed tomography scanner comprising: scanning an object in a cone-beam imaging geometry following a general triple helix path wherein projection data is generated: reconstructing the image, wherein the reconstructing comprises performing a filtered backprojection; using a fast exact or quasi-exact filtered back projection algorithm to generate the backprojected data; and using the backprojected data to generate an image with improved temporal resolution.

U.S. patent application Ser. No. 12/916,458, filed Oct. 29, 2010, and entitled “Tomography-Based and MRI-Based Imaging Systems,” discloses various imaging techniques, such as clinical x-ray CT, optical molecular tomography, multi-scale/parameter X-ray CT, dynamic cardiac elastography, and exact and stable interior ROI reconstruction for radial MRI. U.S. patent application Ser. No. 12/938,303, filed Nov. 2, 2010 and entitled “Methods for Improved Single Photon Emission Computed Tomography Using Exact and Stable Region of Interest Reconstructions,” discloses additional techniques applicable to CT type imaging modalities. Still further, U.S. patent application Ser. No. 12/945,733, filed Nov. 12, 2010 and entitled “Extended Interior Methods and Systems for Spectral, Optical, and Photoacoustic Imaging,” discloses for example a system for image reconstruction comprising: multiple sources for emitting x-rays to pass through a region of interest (ROI) at multiple orientations; a detector array for receiving overlapping x-ray projection data from the multiple sources; a processing module operably configured for: receiving the overlapping x-ray projection data; and reconstructing the ROI into an image by: determining a difference between data relating to a first actual image and data relating to a second expected image of higher resolution than the first image; iteratively updating the expected data and iteratively updating the corresponding difference between the actual and expected data; performing a Taylor series expansion to linearize the Imaging system by omitting high order terms; and performing POCS-gradient algorithm on the linearly approximated system iteratively.

When compared with CT, MRI is distinguished by its superior contrast resolution. Unlike CT imaging, MRI does not involve ionizing radiation and has multiple contrast mechanisms: proton density weighted, T1 or T2 weighted, contrast-enhanced, motion sensitive, elastic, temporal, and chemical shift imaging. See Bammer, R., Basic principles of diffusion-weighted imaging. European journal of radiology, 2003. 45(3): p. 169-184; Gatehouse, P. O. and G. M. Bydder, Magnetic Resonance Imaging of Short T2 Components in Tissue. Clinical Radiology, 2003. 58(1): p. 1-19; Haase, A., Snapshot flash mri, applications to t1, t2, and chemical-shift imaging. Magnetic Resonance in Medicine, 1990. 13(1): p. 77-89; Le Bihan, D., et al., MR imaging of intravoxel incoherent motions: application to diffusion and perfusion in neurologic disorders. Radiology, 1986. 161(2): p. 401-407; Nishimura, D. G., Time-of-flight MR angiography. Magnetic Resonance in Medicine, 1990. 14(2): p. 194-201; R. Muthupillai. D. J. L., P. J. Rossman, J. F. Greenleaf, A. Manduca, R. L. Ehman, Magnetic resonance elastography by direct visualization of propagating acoustic strain waves. Science, 1995. 269: p. 1854-1857; R. Muthupillai, R. L. E., Magnetic resonance elastography. Nature Medicine, 1996. 2: p. 601-603. However, MRI is generally considered significantly slower and more expensive than CT. In addition, scanning patients with certain implantable devices is contraindicated unless the device is MRI compatible. See TO, W., Guidance for Industry and FDA Staff—Establishing Safety and Compatibility of Passive Implants in the Magnetic Resonance (MR) Environment, in http://www.fda.gov/cdrh/osel/guidance/1685.html Aug. 21, 2008.

PET has the advantages of high sensitivity and specificity with moderate spatial resolution (one to several millimeters). See Karp, G.M.a.J.S., Positron emission tomography Phys. Med. Biol., 2006. 51: p. R117-R137; Phelps, M. E., et al., Application of Annihilation Coincidence Detection to Transaxial Reconstruction Tomography. J Nucl Med, 1975. 16(3): p. 210-224; and Manning. K., at al., Clinical Practice Guidelines for the Utilization of Positron Emission Tomography/Computed Tomography Imaging in Selected Oncologic Applications: Suggestions from a Provider Group. Molecular Imaging and Biology, 2007. 9(6): p. 324-332. It is heavily used in clinical oncology, brain mapping, neurologic and cardiac functional imaging. While CT and MRI isolate anatomic and functional changes, PET (and SPECT) are capable of detecting molecular activities, even prior to phenotype expressions. See Aarsvold, M.N.W.a.J.N., Emission Tomography: The Fundamentals of PET and SPECT, 2004: Elsevier Academic Press; and Nestle. U. and et al., Biological imaging in radiation therapy: role of positron emission tomography. Physics in Medicine and Biology, 2009. 54(1): p. R1. PET uses injected radiochemical probes (positron emitters), with differential uptake rates modulated by tissue vasculature and metabolism, and takes longer scan time (˜30 minutes) than CT and MRI. Quantitative information can be extracted from PET. One limitation to its widespread use is the high cost of producing its short-lived radionuclides and radiopharmaceutical probes.

SPECT, along with PET, depends on radioactive probes and resultant gamma rays. See Knoll, G. F., Single-photon emission computed tomography. Proceedings of the IEEE, 1983. 71(3): p. 320-329; and Mullan B P, O.C.M., Hung J C., Single photon emission computed tomography. Neuroimaging Clin N Am., 1995. 5(4): p. 647-673. In contrast to PET, SPECT probes emit gamma photons that are directly measurable, whereas PET probes emit positrons that annihilate with electrons to form a pair of measurable collinear gamma photons. Generally, SPECT has a lower spatial resolution (about 10 mm) and a longer scan time (several hours) than PET, but it is significantly less expensive than PET, and is important for cardiac, brain, tumor, infection, thyroid or bone imaging.

Ultrasound (US) imaging stands alongside CT, MRI, PET and SPECT, and has profoundly impacted the medical practice due to its portability, safety, cost effectiveness, and real-time performance. See Szabo, T. L., Diagnostic Ultrasound Imaging: Inside Out. 2004: Elservier Academic Press. However, medical US is limited by the high attenuation and complexity of acoustic interaction with bone and air. Thus, it is primarily useful for examining the outer surface of soft structures and organs. US images can be enhanced with contrast agents such as micro-bubbles. On the other hand, focused US can serve therapeutic purposes.

Optical imaging depends on optical parameters and/or light probes. See Hebden, S.R.A.a.J.C., Optical imaging in medicine: II. Modelling and reconstruction. Physics in Medicine and Biology, 1997. 42(5): p. 841-853. Diffuse optical tomography (DOT) reconstructs distributions of tissue absorption and scattering coefficients, and has applications in breast imaging etc. Enabled by fluorescence probes, optical fluorescence imaging can sense biological processes in vivo at the cellular and molecular levels. Of particular interest, fluorescence tomography employs fluorescence signals, induced by laser light or x-rays, to determine a volumetric distribution of fluorescent probes. As a prerequisite, anatomical structures and optical properties of the tissue need to be estimated.

A summary of these imaging modalities with their individual strengths and weaknesses is provided below in Table I.

TABLE I
Comparison of medical imaging modalities CT, MRI,
PET, SPECT, US imaging and optical imaging.

| | Spatial/ | | | |
|---|---|---|---|---|
| Imaging | Temporal | Imaging | | |
| Modality | Resolution | Mechanism | Strengths | Drawbacks |
| CT | 10 nm- | X-ray | High spatial/ | Ionizing |
| | 1 mm/ | atten- | temporal | radiation, |
| | 10 ms | uation | resolution, | low soft-tissue |
| | | | high bony | contrast, less |
| | | | contrast | functional and |
| | | | | biochemical |
| | | | | information |
| | | | | content |
| MRI | 100 μm- | Magneti- | High soft-tissue | Lower spatial |
| | 5 mm/ | zation | contrast, rich | and temporal |
| | 1 s | of atoms | functional, | resolution, |
| | | | biochemical, and | body device |
| | | | metabolism | and implants |
| | | | information | incompatibility |
| | | | content | |
| PET | 1 mm/ | Positron- | High sensitivity | Lower spatial |
| | 100 s | labeled | and specificity, | and temporal |
| | | ligand | rich functional, | resolution, |
| | | binding | biochemical, and | partial-volume |
| | | | molecular | effect, high |
| | | | information | noise |
| | | | content | |
| SPECT | 1 mm- | Gamma- | High sensitivity | Low spatial |
| | 10 mm/ | emitting | and specificity, | and temporal |
| | 100 s | radioligand | rich functional, | resolution, |
| | | binding | biochemical, and | partial-volume |
| | | | molecular | effect, high |
| | | | information | noise |
| | | | content | |
| US | 100 μm- | Acoustic- | Real-time | Difficult |
| | 1 mm/ | tissue | imaging, | through |
| | 10 ms | interaction | compact and | air and bone, |
| | | | portable | limited field of |
| | | | | view, less |
| | | | | functional and |
| | | | | biochemical |
| | | | | information |
| | | | | content |
| Optical | 1 mm- | Target- | Compact, rich | Limited |
| | 10 mm/ | seeking | molecular | penetration, |
| | 1 s | light- | information | poor spatial |
| | | emitting | content | and temporal |
| | | probing | | resolution |

Since the advent of diagnostic imaging, there has been a powerful push to combine imaging modalities in a single global coordinate system. The results are widely used multimodality commercial systems: PET-CT, SPECT-CT PET-MRI, and others. These systems represent the cutting-edge of imaging technology, are generally well received and have greatly facilitated important biomedical imaging tasks. See Bockisch 2009; Delbeke 2009; Even-Sapir 2009; Boss, A., et al., Hybrid PET/MRI of Intracranial Masses: Initial Experiences and Comparison to PET/CT. J Nucl Med, 2010: p. jnumed. 110.074773 (“Boss 2010”); Boss, A., et al., Feasibility of simultaneous PET/MR imaging in the head and upper neck area. European Radiology, 2011: p. 1-8 (“Boss 2011”); Bouchelouche. K., et al., PET/CT Imaging and Radioimmunotherapy of Prostate Cancer. Seminars in Nuclear Medicine, 2011. 41(1): p. 29-44; Brunetti, J., et al., Technical Aspects of Positron Emission Tomography/Computed Tomography Fusion Planning. Seminars in Nuclear Medicine, 2008. 38(2): p. 129-136; Cherry, S. R., Multimodality Imaging: Beyond PET/CT and SPECT/CT. Seminars in Nuclear Medicine, 2009 (“Cherry 2009”). 39(5): p. 348-353; Scharf, S., SPECT/CT Imaging in General Orthopedic Practice. Seminars in Nuclear Medicine, 2009. 39(5): p. 293-307; Seo, Y., C. Mari, and B. H. Hasegawa, Technological Development and Advances in Single-Photon Emission Computed Tomography/Computed Tomography. Seminars in Nuclear Medicine, 2008. 38(3): p. 177-198; Townsend, D. W., Positron Emission Tomography/Computed Tomography. Seminars in Nuclear Medicine, 2008. 38(3): p. 152-166; Townsend, D. W., Multimodality imaging of structure and function. Physics in Medicine and Biology, 2008. 53(4): p. R1.

Indeed, since the seminal paper by Dr. Bruce Hasegawa (see Bruce H. Hasegawa, E. L. G., Susan M. Reilly, Soo-Chin Liew and Christopher E. Cann, Description of a simultaneous emission-transmission CT system. 1990 (Proc. SPIE 1231), subsequent work on combining emission and transmission tomography has led to powerful hybrid imaging capabilities that have had major impacts on biomedical applications. The popularity of these hybrid modalities is a testament to the important value of tight association between anatomical information from CT and functional information from SPECT/PET. The first PET-CT system was developed in the late 1990's (see Beyer T, T. D., Brun T, et al., A combined PET/CT scanner for clinical oncology. J Nucl Med., 2000.41: p. 1389-1379), and has been subsequently applied in the biomedical field to study various forms of cancer (e.g. brain, lung, thyroid, lymph, head and neck cancer), along with infection, inflammation and others. Several commercial PET-MRI scanners have been introduced over the past few years and are demonstrating promising results (see Boss 2010 and Boss 2011). The power of the PET-MRI system lies in the synergy between PET and MRI. PET is capable of quantifying myocardial blood flow, assessing viability and prognosis, and is sensitive to many biomarkers of inflammation, angiogenesis, nervous functions, and therapeutic genes or cells. MRI determines ventricular function and structure, and also delineates infarction using a chelated-gadolinium enhancement agent. Another advantage of PET-MRI is its 4D imaging ability that allows for better contrast enhancement of small lesions through motion correction.

Other modality fusion systems include the bioluminescence tomography (BLT) and micro-CT system developed by Dr. Wang's group in 2002, optical and MRI systems, hybrid nuclear and optical imaging systems, x-ray and MRI systems, photoacoustic tomography scanners, and more. See Jiang, M., et al., Image reconstruction for bioluminescence tomography from partial measurement. Opt Express, 2007. 15(18): p. 11095-116: Cong. W., et al., Practical reconstruction method for bioluminescence tomography. Opt Express, 2005. 13(18): p. 6756-71; Wang. G., Y. Li, and M. Jiang, Uniqueness theorems in bioluminescence tomography. Med Phys, 2004. 31(8): p. 2289-99; and Ntziachristos, V., et al., Looking and listening to light: the evolution of whole-body photonic imaging. Nat Biotechnol, 2005. 23(3): p. 313-20.

PET-CT revolutionized medical diagnosis. A PET-CT scanner sequentially acquires patient PET and CT images in an integrated gantry. As a result, functional information (e.g. metabolic or biochemical processes) obtained from PET can be accurately co-registered with anatomic information from CT. As such, PET-CT has forever changed the fields of oncology, surgical planning, radiation therapy, and others so much that most imaging centers have upgraded from conventional PET to PET-CT.

Examples of contemporary modality fusion are not rare. The first human PET, SPECT and CT system (see http://www.mediso.de/anyscan-sc.html) was developed by Mediso, referred to as AnyScan. It provides sequential anatomical and functional imaging within a single framework, accommodating all images with motion artifact suppression and attenuation background correction. Another company, Carestream, has put seven preclinical imaging modalities in two instruments covering PET, SPECT, CT, fluorescence, luminescence, radioisotopic and radiographic imaging. See http://www.cmi-marketing.com/7modalities. Likewise, the Carestream Albira system is designed for sequential micro-PET, micro-SPECT and micro-CT data acquisitions and global reconstructions, similar to the Mediso AnyScan system.

PET-MRI is the most recent frontier in modality fusion. See Boss 2010; Boss 2011; and Pichler, B. J., et al., Positron Emission Tomography/Magnetic Resonance Imaging: The Next Generation of Multimodality Imaging?Seminars in Nuclear Medicine, 2008. 38(3): p. 199-208. PET/MRI delivers molecular information (e.g., cell surface reactors, enzymes and gene expression) from PET, along with anatomical and functional data from MRI. Hence, PET/MRI is capable of assessing flow, diffusion, perfusion and cardiac motion in a single examination, and evaluating myocardial viability (PET) and metabolism (MRI) for diagnosis of cardiac pathology, such as coronary artery disease.

Medical imaging has a huge potential to support the physiome project with a yet-to-be-much-improved capability of obtaining functional information at multi-levels from organs to physiological units. The physiome concept was first presented to the International Union of Physiological Sciences (IUPS) in 1993, and later designated as a strategic area by IUPS in 2001. See Hunter, P., P. Robbins, and D. Noble, The IUPS human physiome project. Pflügers Archiv European Journal of Physiology, 2002. 445(1): p. 1-9; Hunter, P., et al., Integration from proteins to organs: the IUPS Physiome Project. Mechanisms of Ageing and Development, 2005. 126(1): p. 187-192; and Hunter, P. J. and T. K. Borg, Integration from proteins to organs: the Physiome Project. Nat Rev Mol Cell Biol, 2003. 4(3); p. 237-243. Physiome describes physiological processes and their interactions from the genome scale to complex organism in a systematic fashion. The IUPS Physiome Project supports a worldwide repository of models and datasets, and represents an integral component of systems biology and modern medicine.

Likewise, efforts are being made to link molecular assays with diagnostic imaging. See Rutman, A. M. and M. D. Kuo, Radiogenomics: Creating a link between molecular diagnostics and diagnostic imaging. European Journal of Radiology, 2009. 70(2): p. 232-241; and van Houten, V. M. M., et al., Molecular Assays for the Diagnosis of Minimal Residual Head-and-Neck Cancer: Methods, Reliability, Pitfalls, and Solutions. Clinical Cancer Research, 2000. 6(10): p. 3803-3816. Success to date, however, has been rather limited. One reason is that current medical imaging scanners do not individually offer a wide enough spectrum of information. Current x-ray CT scanners produce gray-scale images of physiological or pathological process. On the other hand, large amounts of information from genetic and epigenetic profiling is becoming available. This imbalance between phenotype information (e.g., CT images) and genome-level information (e.g., RNA data) demands more features from the in vivo imaging side.

Indeed, the medical imaging field is rapidly trending in this direction. Turning again to x-ray CT, we see transition from gray-scale to true-color images with development of energy-sensitive, photon-counting detector technology. Another area of advancement is x-ray phase-contrast and dark-field imaging. Overall, imaging modalities and contrast agents are constantly being improved to generate increasingly more information about structural, functional, cellular and molecular characteristics.

What current biomedical imaging systems lack, however, is the ability to provide an integrated system capable of producing simultaneous, dynamic observations in vivo of highly complex biological phenomena. Current multimodal fusion approaches have made significant strides towards meeting this challenge, as demonstrated by the popularity of PET-CT and other hybrid systems. See Ahn, P. H. and M. K. Garg, Positron Emission Tomography/Computed Tomography for Target Delineation in Head and Neck Cancers. Seminars in Nuclear Medicine, 2008. 38(2): p. 141-148 (“Ahn 2008”); Bockisch, A., et al., Hybrid Imaging by SPECT/CT and PET/CT: Proven Outcomes in Cancer Imaging. Seminars in Nuclear Medicine, 2009. 39(4): p. 276-289 (“Bockisch 2009”); Delbeke, D., et al., Hybrid Imaging (SPECT/CT and PET/CT): Improving Therapeutic Decisions. Seminars in Nuclear Medicine, 2009. 39(5): p. 308-340 (“Delbeke 2009”); Even-Sapir, E., Z. Keidar, and R. Bar-Shalom, Hybrid Imaging (SPECT/CT and PET/CT)—Improving the Diagnostic Accuracy of Functional/Metabolic and Anatomic Imaging. Seminars in Nuclear Medicine, 2009. 39(4): p. 264-275 (“Even-Sapir 2009”); and Kaufmann. P. A. and M. F. Di Carli, Hybrid SPECT/CT and PET/CT imaging: The Next Step in Noninvasive Cardiac Imaging. Seminars in Nuclear Medicine, 2009. 39(5): p. 341-347 (“Kaufmann 2009”). As the number of modalities used for simultaneous data acquisition increases, so does the complexity of acquiring the underlying data.

Combining multiple modalities, especially three or more imaging modalities, for the purpose of simultaneous data acquisition is not a trivial task. Due to the bulkiness of the instruments involved and limited space, a “plug and play” type strategy for merely placing the instruments side by side is inadequate and would result in the less desired result of sequential data acquisition. What is needed is a re-working of the technology incorporated into these instruments to provide improved interior tomographic reconstruction techniques which would enable more compact modalities.

### SUMMARY OF THE INVENTION

To this end, the inventors provide for integration of multiple modalities into a single scanner for truly simultaneous and information-rich data acquisition. In embodiments, three or more imaging modalities, including CT. MRI, PET, SPECT, US imaging, optical imaging, and more, are integrated into a single scanner.

One object of the invention is to target the tightest possible integration of all imaging modalities, and methodically fuse the richest relevant information available from each technology. This target can be reached by applying the latest insights from interior tomography and compressive sensing principles to design an omni-tomography system, a system that places the highest demands on the broadest array of known imaging hardware and systems engineering. The logic seems clear that since subsets of imaging modalities are synergistic, the integration of all imaging modalities as a whole should, in principle, add values above that of the individual sets.

Major technical obstacles to omni-tomographic systems include gantry space limitation, the associated technical difficulties and high cost. An immediate advantage of interior imaging can be seen in how the problem of SPECT-MRI must be addressed. SPECT-MRI has two unique issues: interference to the magnetic field caused by a rotating camera head, and the induction of eddy currents in the camera head itself. Fortunately, interior SPECT reconstruction can effectively address these issues simultaneously. With a smaller SPECT camera, the electromagnetic interference can be reduced, and electromagnetic shielding design can be simplified. Eddy currents can be diminished in significance with proper shielding and smaller detector heads.

It is underlined that, in a substantial sense, omni-tomography covers what is known today as “modality fusion”. With flexibility at a clinician's discretion, according to a proper protocol, the inventive omni-tomography scanner allows use of a single modality or multiple modalities in simultaneous combination on an ad-hoc basis. For a diagnosis/treatment where the patient requires multiple scans from various imaging modalities, omni-tomography could be a cost-effective way in terms of space utilization, equipment costs and patient throughput. In a sense, omni-tomography could be a versatile cost effective imaging platform, comparable to a fully-fledged medical imaging center. The costs for startup and implementation of an omni-tomography system could be significantly lower than a large-scale hospital imaging department.

Another tomography system according to embodiments of the invention includes a focus on a relatively small region of interest (ROI), the synergy of relatively more imaging modes of a different nature, and the compactness of the resultant systems. An interior x-ray CT imaging chain (an x-ray source and an x-ray detector array) can be orthogonally integrated with an interior SPECT imaging chain (one small cone-beam angle SPECT collimator) to form an interior CT/SPECT system.

Specific embodiment of systems according to the invention can include an omni-tomography system comprising two or more imaging modalities operably configured for concurrent signal acquisition for performing ROI-targeted reconstruction. Preferably, such systems comprise three or more imaging modalities.

The imaging modalities incorporated into the omni-tomography systems of the invention can be one or more modality chosen from CT, MRI, PET, SPECT, Ultrasound and optical imaging subsystems. Preferably, the imaging modalities are capable of performing image reconstruction based on interior techniques.

Systems and devices according to embodiments of the invention can comprise a single gantry in a ring or O-shaped configuration. Preferred embodiments can have three concentric rings: a first inner ring as a permanent magnet; a second middle ring containing an x-ray tube, detector array, and a pair of SPECT detectors; and a third outer ring for containing PET crystals and electronics.

Such systems and devices can be operably configured such that the inner and outer rings are static. Preferably, the second middle ring is operably configured to rotate and acquire data for interior CT and interior SPECT.

In embodiments, the second middle rotating ring is embedded in a slip-ring which supports the rotating ring and facilitates power/signal transmission.

Even further, in such systems and devices the second middle rotating ring, the slip-ring, and the third outer PET ring rotate through magnetic poles.

The systems and devices can comprise a yoke for N and S magnetic poles which yoke is configured as a C-shaped arm.

Size of the gantry is not critical. The gantry can be configured for a standard sized patient or smaller. Preferably, the systems and devices of the invention are operably configured for human or animal subjects. Most preferably, such systems and devices are operably configured for accommodating a patient of up to approximately 170 cm in height, 70 kg, with a chest size of 22 cm in AP direction and 35 cm in lateral direction.

According to embodiments, the MRI subsystem comprises two permanent magnet heads at each magnetic pole.

It is preferred that the MRI subsystem is operably configured for providing a magnetic field for a ROI of about 15-20 cm at a center point of the gantry, with a vertical gap between magnet poles in the range of about 30-70 cm and with magnet heads about 20-60 cm in width and about 40-120 cm in length. Smaller or larger ROIs can be targeted and the systems and devices operably configured appropriately. The MRI subsystem can provide a magnetic field for a ROI of about 0-50 cm, such as about 30-45 cm, or from about 1-5 cm, or further from about 3-10 cm. The magnet poles can be separated by a vertical distance of about 5-200 cm, depending on the overall size of the gantry and the expected size of the patient or subject.

Systems and devices of the invention can have solid or hollow magnet heads, with preferably hollow magnet heads.

The CT subsystem in preferred embodiments has an x-ray source and opposing x-ray detector array with a source-to-detector distance in the range of about 60-100 cm. Again the separation distance will be highly dependent on the overall size of the gantry and can be configured according to the expected size of the patient. For smaller or larger systems the source-to-detector distance can be in the range of about 5-200 cm, such as from about 10-150 cm, or from about 20-125 cm, such as from about 30-90 cm, or from about 50-80 cm, or 75 cm.

Preferably, the CT subsystem is operably configured to acquire data for interior CT and for compressive sensing based image reconstruction.

The SPECT detectors can be collimated to parallel-beam geometry and arranged orthogonally. Additionally, the SPECT detectors are solid-state CZT SPECT detectors in various embodiments.

Exemplary systems and devices of the invention can comprise CZT SPECT detectors that are operably configured for detecting x-ray and gamma-ray photons simultaneously.

The SPECT subsystem can comprise a converging, diverging, or pinhole collimator, such as for example a multi-pinhole collimator.

In embodiments, the third outer ring has an internal diameter in the range of about 80-200 cm and comprises a PET detector with LYSO crystals or solid-state materials or CZT. Size of the outer ring, and each ring, will be dependent on the overall size of the gantry as well as the requirements for each of the other modalities and the corresponding size of the ring containing those modalities. The PET ring can be in the range of about 20-200 cm, such as about 30-140 cm, including from about 50-100 cm, or from about 70-90 cm, and so on.

The PET detector is preferably operably configured for performing PET reconstruction using an adapted interior tomography algorithm.

The ultrasound (US) subsystem preferably comprises a US transducer operably configured for disposition on a physiologically relevant ROI of a patient.

Systems and devices of the invention can also comprise a photo-acoustic imaging modality.

In preferred embodiments, there is an optical imaging subsystem comprising an x-ray luminescence or x-ray fluorescence camera.

Even further, the systems and devices can comprise an interior x-ray fluorescence CT imaging modality.

The MRI subsystem in embodiments comprises permanent magnets for providing a homogeneous or inhomogeneous local magnetic field.

Methods of using the omni-tomography systems and devices described in this specification are also included as embodiments. For example, a method of interior MRI of a Region of Interest (ROI) based on a locally homogeneous or an inhomogeneous magnetic background field is within the scope of the invention and can be used as a stand-alone MRI or can be incorporated into omni-tomography instruments of the invention. Systems using such methods are also included within the scope of the invention and for example additionally comprise the requisite imaging hardware (e.g., MRI, CT, SPECT, PET, etc., imaging hardware), a processing module (software) for performing the modality-specific imaging methods, and a computer processor in operable communication with the processing module for executing the methods of the processing module.

Such MRI systems and subsystems of the invention can comprise software for imaging a ROI operably configured for: acquiring MR signal data by tuning a radiofrequency (RF) pulse to excite any iso-region of the ROI or by using compressive sensing acquisition; recording the MR signal data for each iso-region; further localizing each iso-region into desired voxels using x-, y- and z-linear gradient fields that can be either time-invariant or time-varying; and reconstructing an image of the ROI, wherein the ROI D is represented by a union of constant intensity iso-regions of the local magnetic field B₀ along the z-direction:

$$D = {⋃}_{c} ( \overline{x} \text{:} {B}_{0} ( \overline{x} ) = c \} , c ∈ \{ {B}_{min} , {B}_{max} \} , , \qquad ( III . B .1 )$$

where \vec{x} denotes a spatial point.

Such systems, devices and methods can also comprise or be operably configured to provide for: exciting each iso-region with an RF pulse having an excitation and demodulation frequency that is the same as the resonance frequency for the target iso-region, such that to excite the iso-region with a radius r=r₀, the resonance frequency is: ω₀(r,θ)=γβ₀δ(r−r₀), (III.B.3) and the demodulation frequency is proportional to: s(t)=∫ω₀ρe^{iγ(xG}^{ₓ}^{+yG}^{_{y}}^{)t}dxdy, (III.B.4), where ρ represents a 2D MR image to be reconstructed; and reconstructing the iso-region at r=r₀ using the Inverse Fourier Transform; and continuing reconstruction in this manner to recover all iso-regions and reconstruct an entire image of the ROI.

Additional embodiments of the invention include a method of interior MRI of a Region of Interest (ROI) based on an inhomogeneous magnetic background field comprising: randomizing gradient field orientation indexes and iso-curve indexes and sequentially pairing field orientations with iso-curves; performing compressive sensing acquisition of data relating to an ROI by sampling each iso-curve under only one gradient orientation, wherein gradient fields (Gₓ, G_{y}) by (G, θ₀) are represented as follows: Gₓ=G cos θₐ, G_{y}=G sin θ_{G}, θ₀ε[0,π], (III.B.5), where the orientation angle % is random but fixed for a given iso-curve; separating non-unique spatial locations on the iso-curve to satisfy image smoothness conditions; and reconstructing each iso-curve and obtaining an entire image of the ROI.

Even further, Included is a method of interior x-ray fluorescence tomography comprising: disposing gold or nano-phosphor nanoparticles in a ROI of a body or tissue; and performing X-ray fluorescence computed tomography on the ROI to map disposition of the nanoparticles.

### BRIEF DESCRIPTION OF THE DRAWINGS

The accompanying drawings illustrate certain aspects of some of the embodiments of the present invention, and should not be used to limit or define the invention. Together with the written description the drawings serve to explain certain principles of the invention.

FIG. 1A is a schematic drawing illustrating an omni-tomographic imaging system according to embodiments of the invention.

FIG. 1B is a schematic drawing illustrating another omni-tomographic imaging system according to embodiments of the invention.

FIGS. 2A, 2B and 2C are schematic diagrams showing a representative magnetic field that can be generated using the MRI component of the omni-tomographic system.

FIG. 3 is a schematic diagram illustrating a projection model with a finite detector element and a finite focal spot in fan-beam geometry.

FIG. 4 is a schematic diagram illustrating a discrete projection model assuming a discrete image in fan-beam geometry.

FIGS. 5A, 5B and 5C are images showing an interior reconstruction according to embodiments of the invention.

FIGS. 5D and 5E are profiles along the horizontal and vertical white lines in FIGS. 5B-5C, respectively, where the thick lines on the horizontal axis indicate the ROI.

FIGS. 6A and 6B are schematic diagrams showing pulse sequences for interior MRI, where FIG. 6A is an illustrative sequence for 3D imaging, and FIG. 6B is a sequence for slice-based imaging.

FIGS. 7A, 78 and 7C are schematic diagrams showing selective z-slice excitation by a dynamic z-gradient field.

FIGS. 8A, 8B and 8C are images of a global MRI reconstruction.

FIG. 9 is images showing MR data acquired within an ROL

FIG. 10 is a schematic diagram illustrating interior SPECT imaging in parallel-beam geometry.

FIG. 11A is an image of an original SPECT ROI image.

FIG. 11B is an image showing interior reconstruction using the HOT minimization algorithm.

FIGS. 11C and 11D are graphical representations of the pseudo-color counterparts of FIGS. A-B respectively.

FIGS. 11E and 11F are graphs showing the representative profiles along the white (FIG. 11E) horizontal and (FIG. 11F) vertical lines respectively.

FIG. 12 is a schematic diagram of XFCT.

FIGS. 13A, 13B and 13C are various figures relating to interior XFCT reconstruction and more particularly, FIG. 13A is an image showing the true distribution of golden nanoparticles accumulated in four circular regions overlapped on a CT image; FIG. 13B is an image of the reconstructed interior nanophosphor distributions in the ROI; and FIG. 13C is a graph showing the profiles through the oblique line of 480.

### DETAILED DESCRIPTION OF VARIOUS EMBODIMENTS OF THE INVENTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

To go beyond existing modality fusions, it might be imagined that additional modalities should be added for simultaneous characterization of additional biomedical properties. See Cherry 2009; Mah, D. and C. C. Chen, Image Guidance in Radiation Oncology Treatment Planning: The Role of Imaging Technologies on the Planning Process. Seminars in Nuclear Medicine, 2008. 38(2): p. 114-118; and Patton, J. A., D. W. Townsend, and B. F. Hutton, Hybrid Imaging Technology: From Dreams and Vision to Clinical Devices. Seminars in Nuclear Medicine, 2009. 39(4): p. 247-263. This mission may appear impractical, however, due to space conflict with the physical size requirements of current scanners and other physical constraints. Additional scanners could be assembled longitudinally, but this sequential arrangement would make synchronized capture impossible, especially when relatively slow modalities are involved (e.g. PET and SPECT). The arguments for and limitations of the classic modality fusion approach are demonstrated in the latest development of the Advanced Multimodality Image Guided Operating (AMIGO) Suite project, which was the first multimodality suite in the world to give surgeons and interventional specialists immediate access to a full array of imaging modalities for use during procedures.

To overcome the aforementioned challenge for grand fusion, interior tomography can be elevated from a specific imaging mode to a general guiding principle for the biomedical imaging field. Over the past years, interior tomography has been studied for theoretically exact CT image reconstruction over an internal region of interest (ROI) from data associated only with lines through the ROI, which means that a relatively narrow imaging chain can be made. See G Wang, H. Y., Y B Ye, A scheme for multisource interior tomography. Medical Physics, 2009. 36(8): p. 3575-3581; Jiansheng Yang, H. Y., Ming Jiang and Ge Wang, High-order total variation minimization for interior tomography. Inverse Problems, 2010. 3(26): p. 1-29; and Wang, H.Y.a.G., Compressed sensing based interior tomography. Physics in Medicine and Biology, 2009. 54(9): p. 2791-2805. This approach has been extended for interior SPECT (see Yu H Y, Y. J., Jiang M, Wang G, Interior SPECT—Exact and stable ROI reconstruction from uniformly attenuated local projections. Communications in Numerical Methods in Engineering, 2008: p. 18 pages) and also achieved some success for interior MRI (see J. Zhang, H. Y., C. Corum, M. Garwood, G. Wang Exact and Stable Interior ROI Reconstruction for Radial MRI. Proc. SPIE, 2009. Vol. 7258: p. 8).

Omni-tomography is inspired and enabled by a recent breakthrough —interior tomography. Since 2007 the inventors have been developing interior tomography for precise reconstruction over an ROI from purely local data. See Wang G, Ye Y, Yu H Y: Interior tomography and instant tomography reconstruction from truncated limited angle projection data. U.S. Pat. No. 7,697,658 B2, Apr. 13, 2010: Ye Y, Yu H Y, Wei Y, Wang G: A general local reconstruction approach based on truncated Hilbert transform. Intentional Journal of Biomedical Imaging ID 63634. 2007: Wang G. Yu H Y, Ye Y B: A scheme for multi-source interior tomography. Medical Physics 36:3575-3581, 2009; Yu, H Y., Wang G: Compressed sensing based Interior tomography. Phys Med Biol 54:2791-2805, 2009; Yang J S, Yu, H Y, Jiang M. Wang G: High-order total variation minimization for interior tomography. Inverse Problems 26:035013, 2010; Wang G, Yu H Y: Can interior tomography outperform lambda tomography?Proc Natl Acad Sci 107:E92-3, 2010; and Katsevich E, Katsevich A, Wang G: Stability of interior tomography. In review, 2011. While classic CT theory targets theoretically exact reconstruction of a whole cross-section from untruncated projections, real-world applications often focus on a small region of interest (ROI). A long-standing difficulty has been that traditional CT methods cannot exactly reconstruct an ROI solely from truncated projections along x-rays through the ROI. This interior problem was studied for decades, and the fact that precise reconstruction cannot be obtained from local data contributed to the long-standing CT architectures whereby detectors always fully cover a transaxial slice.

The inventors overcome these limitations of the art by compressing each imaging modality into a slim imaging chain for ROI-targeted reconstruction, instead of traditional global reconstruction, and use a single gantry system for truly concurrent signal acquisition and composite interior reconstruction in a unified framework, regularized by prior knowledge from sparsity to atlases. This is referred to in the context of this specification as omni-tomography or multi-tomography.

Omni-tomography has the potential to open up exciting new opportunities in research, development and applications. Specifically, it may offer temporally-correlated quantitative physiological, pathological, pharmacokinetic and pharmacodynamic information. Examples include: metabolic processes, enzyme activities, protein bindings, kinetic interplays, tumor & stem cell tracking, longitudinal studies in cardiology, oncology, neuroscience, tissue engineering, and so on. See Ge Wang, Jie Zhang, Hao Gao, Victor Weir, Hengyong Yu, Wenxiang Cong, Xiaochen Xu, Haiou Shen, James Bennett, Yue Wang, and Michael Vannier, “Omni-tomography/Multi-tomography
Integrating Multiple Modalities for Simultaneous. Imaging,” submitted for publication 10 Jun. 2011, arXiv:1106.2124v1.

System Architecture.

Given the physical requirements of typical scanners, the primary challenge is to integrate all or many of them into a single gantry. Thanks to the interior tomography principle, there is substantial flexibility to integrate various imaging modalities when they only target a relatively small ROI. The inventors have systematically analyzed a number of architectures and realized that each style has advantages and disadvantages. Although any of these configurations can be used to implement omni-tomography, this specification focuses on a ring-shaped design, or “O” design, which only serves as an initial example of omni-tomography instrumentation.

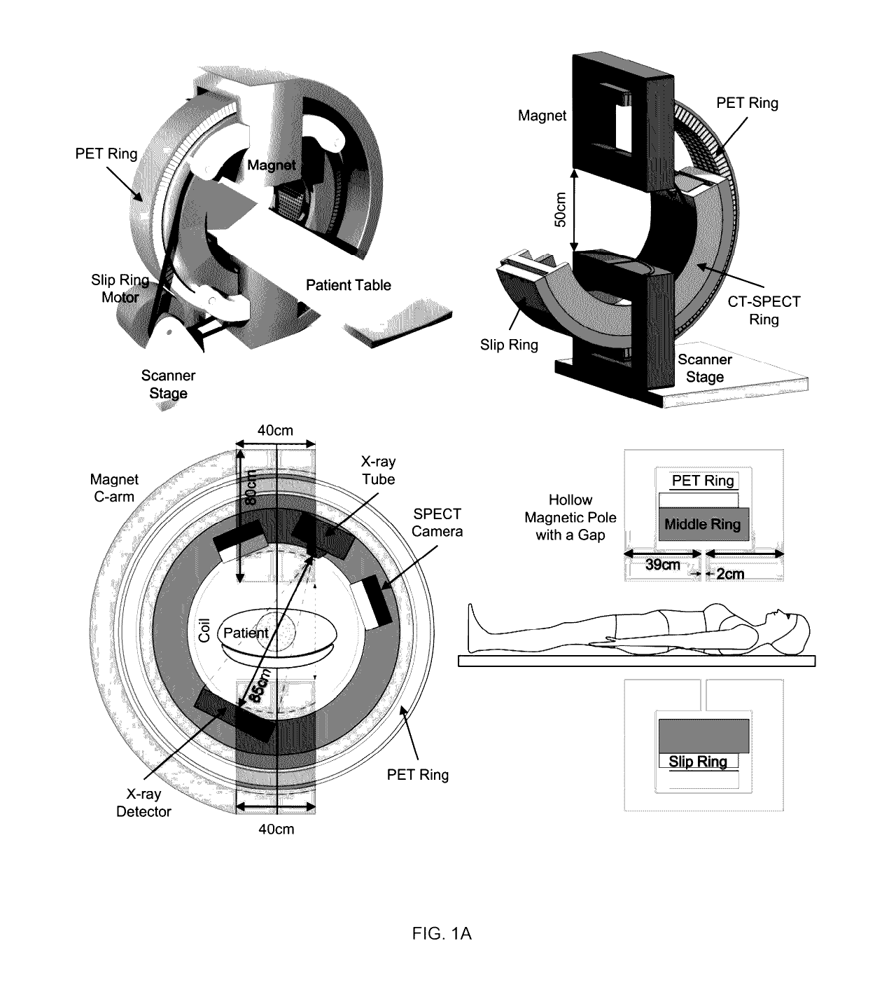

Referring now to FIG. 1A, a schematic diagram is provided illustrating representative system architecture for omni-tomography according to embodiments of the invention. As shown, the exemplary multi-tomography/omni-tomography system architecture can comprise two static rings and one rotating ring for multi-tomography. While the red C-arm is a permanent magnet and the yellow outer ring contains PET crystals, the blue ring supports a CT tube, a CT detector and a pair of SPECT detectors. The blue CT-SPECT ring is on a green slip ring (like a large ball bearing) as the interface for power and data. The CT-SPECT ring, the slip-ring, and the PET ring all go through the magnetic poles. For the first time, the system integrates all major imaging modalities into a conventional gantry space for truly simultaneous acquisition of CT, MRI, PET, SPECT, Ultrasound and optical imaging.

All the major medical tomographic modalities are incorporated into three concentric rings: an inner ring as a permanent magnet; a middle ring containing the x-ray tube and detector array and a pair of SPECT detectors; and an outer ring for PET crystals and electronics (inner and outer rings are preferably static). The middle ring is designed to rotate and acquire data for both interior CT and interior SPECT. This rotating ring is embedded in a slip-ring (similar to a large diameter ball bearing) which supports the rotating ring and facilitates power/signal transmission. The rotating ring, the slip-ring, and the PET ring all go through the magnetic poles. The yoke for N and S poles of the magnet are configured similarly to a “C-arm”. The system is designed for human or animal subjects, and can be configured to accommodate a standard patient size (approximately 170 cm in height, 70 kg, with a chest size of 22 cm in AP direction and 35 cm in lateral direction). Specific preferred features of each imaging modality are provided below.

MRI—

The MRI component is similar to that of a commercial open MRI. All the available techniques for open MRI can be potentially adapted for the omni-tomographic scanner. As shown, in FIG. 1A, the MRI subsystem can consist of two permanent magnet heads at each magnetic pole. The vertical gap between the magnet poles can be about 50 cm and in this case was chosen based on a simulation to provide a sufficient magnetic field for a region of interest (ROI) of approximately 15 to 20 cm in the center of the gantry aperture. This ROI was also used to determine the width (40 cm) and length (2×40 cm) of the magnetic heads. This configuration leaves sufficient space for other modalities to probe the subject without being significantly blocked by the magnet. A deviation from the commercial open MRI design is that each magnet head is hollow and has a gap to let the middle ring modalities “look through” the magnet. Hence, the CT tube and detector, as well as the SPECT cameras, perform full-scans to the extent defined by the gap through the magnet, and cone-beam scans when the magnet is not in the radiation paths. This detailed design for the magnet permits a 2 cm clearance between the two magnet parts of each pole.

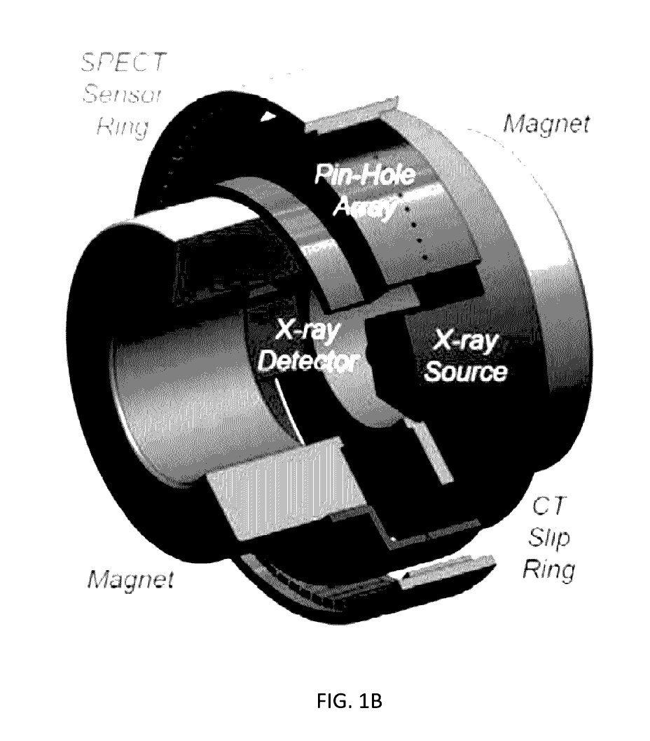

Vizimag (http://www.vizimag.com/) software can be used to simulate the field strength of the magnetic field and realize a locally uniform field between the poles. In a simulation performed to determine optimum parameters for the MRI component of the omni-tomographic system, the desired magnetic field strength was set to 0.2 Tesla. The magnetic field may be adjusted by changing ferromagnetic materials and the dimensions of the magnetic blocks or using an alternative technology. The generated magnetic field is shown in FIG. 2. The magnetic flux varies from 0.208 to 0.211 Tesla over as ROI of 20×20 cm² with its origin at the iso-center of the main imaging plane in the omni-tomographic imager. The field uniformity can be further improved with technical refinements. Further, one of skill in the art will know how to adjust the parameters to achieve a desired effect and indeed any of the MRI systems known in the art can be incorporated into the omni-tomography systems of the invention. Likewise, any of the technical specifications provided in references cited in this specification can be used to obtain an MRI modality compatible with the omni-tomography systems according to the invention.

The gradient coils used with current open MRI scanners can be modified for integration into the inventive omni-tomography gantry. FIG. 1A shows a potential configuration with gradient coils inserted. Generally speaking, any gradient and RF coil settings for open MRI can be used in the inventive systems. See C H Moon, H. W. P., M H Cho and S Y Lee, Design of convex-surface gradient coils for a vertical-field open MRI system. Meas. Sci. Technol., 2000. 11(8): p. N89-N94; and While, P. T., L. K. Forbes, and S. Crozier, 3D gradient coil design for open MRI systems. Journal of Magnetic Resonance, 2010. 207(1): p. 124-133.

MRI shielding is preferred for the architecture, including: radio frequency interference shielding, electromagnetic interference shielding, electromagnetic pulse shielding, and so on. Highly conductive, non-woven electromagnetic shielding materials will be used for this purpose. These materials are usually made from a variety of fibers, such as carbon and nickel-coated carbon, which are flexible enough to accommodate complex contours and shapes. These techniques are well-developed or maturing, and should not pose significant difficulty in principle. See Laskaris, E. T., Open MRI magnet with superconductive shielding. 1995, General Electric Company: United States.

CT—

The middle ring of the omni-tomography system contains the CT and SPECT subsystems. The CT subsystem has an x-ray source and opposing x-ray detector array. A typical source configuration would include an x-ray source (e.g. Varian GS-3074, 23.5×410×13.5 cm³) with a heat exchanger (e.g. Varian HE 300, 23.5×410×13.5 cm³) and a generator (e.g. Spellman 16010, 20.5×40×50 cm³). A flat panel (e.g. Varian PaxScan4030CB, 47×37×7 cm³) or photon-counting spectroscopic detector array may be used. A preferred source-to-detector distance is approximately 85 cm, which is in the range of conventional CT scanners, but can be any distance for example between 10 cm and 200 cm, such as from about 20 cm to about 100 cm, or from about 50 cm to about 70 cm, and so on.

Interior tomography was originally developed for CT, and has already produced impressive results. See Ye; Y., et al., A general local reconstruction approach based on a truncated Hilbert transform. International Journal of Biomedical Imaging, 2007. 2007: p. Article ID: 63634, 8 pages (“Ye 2007”): Yu, H. and G. Wang. Compressed sensing based Interior tomography. Phys Med Biol, 2009. 54(9): p. 2791-2805 (“Yu I 2009”); Yu, H., et al., Supplemental analysis on compressed sensing based interior tomography. Phys Med Biol, 2009. 54(18): p. N425-N432 (“Yu II 2009”); and Yu, H., Y. Ye, and G. Wang, Local Reconstruction Using the Truncated Hilbert Transform via Singular Value Decomposition. Journal of X-Ray Science and Technology, 2008. 16(4): p. 243-251 (“Yu 2008”). Few issues are expected with image reconstruction over an ROI from truncated projection data. Note that the interior CT scan data could be potentially used to estimate the attenuation background over the whole field of view, and enable anatomically-specific attenuation correction for SPECT and PET. In the future, a field emission x-ray source (http://www.xintek.com/products/xray/index.htm) can be used for its dynamic programming capability which is ideally suited for physiological gating and other studies. Also, a grating-based or other coded-aperture-type apparatuses may be used for phase-contrast and dark-field imaging. Time-of-flight x-ray imaging could be a distant possibility. Any of the designs and architectures disclosed in this specification are flexible enough to incorporate these in the future.

SPECT—

Two solid-state SPECT cameras are included in our design. The SPECT detectors are currently collimated to parallel-beam geometry and arranged orthogonally. The dual-detectors should double sensitivity and speed; solid-state CZT SPECT detectors are preferred due to their size and functionality. There are several commercially available systems (Gamma Medica, GE Triumph) that use CZT detectors for molecular Imaging. A CZT detector can potentially detect both x-ray and gamma-ray photons simultaneously, which is great for future development. The 16×20 cm CZT SPECT detector, manufactured by Gamma Medica (Northridge, Calif.), was selected for a preferred embodiment of omni-tomography systems of the invention.

A converging or pinhole collimator may be used within the SPECT subsystem. Both collimators types magnify features in a ROI. A multi-pinhole collimator is an option when better sensitivity is desired. See Boles, C. D., et al., A multimode digital detector readout for solid-state medical imaging detectors. Solid-State Circuits, IEEE Journal of, 1998. 33(5): p. 733-742; and Turner, T. O. C., V. B. Clajus, M. Hayakawa S Volkovskii, A. Multi-Channel Front-End Readout IC for Position Sensitive Solid-State Detectors. Nuclear Science Symposium Conference Record (NSS/MIC), 2006 IEEE Oct. 29, 2006-Nov. 1, 2006. 1: p. 384-388. A diverging collimator can image larger structures with a smaller detector. Furthermore, data compromised when the SPECT cameras are behind the magnetic heads can be fixed by attenuation correction because the fixed magnetic structures are semi-transparent to gamma rays. The rationale for positioning the SPECT and CT subsystems on the middle ring, instead of being in front of the magnetic heads, is to limit Interference from the rotating parts on the magnetic field.

PET—

The PET detector ring of 120 cm internal diameter consists of LYSO crystals, but potentially could be built out of CZT or other suitable sold-state materials. For LYSO crystal-based systems, the resulting scintillation emission can be detected by avalanche photodiode detectors (APDs). The detector units are preferably 4×4×20 mm³. There are 4 units per detector block; 471 detector blocks per ring; and 20 rings in total. The axial extent is 160 mm. Another component is a coincidence timing or time of flight (TOF) analysis circuit, which are commercially available.

The 511 KeV PET photons are sufficiently energetic to pass through the solid-state CT and SPECT detectors and reach the PET detector. The PET circuitry can be customized for ROI imaging. Interior PET reconstruction can be performed using an adapted interior tomography algorithm. Prior knowledge of the CT and SPECT detectors structures can be utilized to conduct hardware-related attenuation correction for PET. Another important consideration is the potential interference from the MRI subsystem that could degrade the performance of the PET subsystem. Positioning the PET ring furthest away from the MRI subsystem should produce maximum isolation for the PET detector and associated electronics from the influence of the magnet field.

In some existing PET-MRI systems, the PET ring is inside the main magnet ring. For example, the Siemens Biograph PET-MRI system incorporates new MRI compatible PET detectors into an MRI system. Many PET-MRI systems use PET inserts for simultaneous PET and MRI. Another system, depicted on a poster from Cambridge University, uses two magnet coils placed end-to-end with a gap in between for the PET detectors. These designs are fundamentally constrained by the global data acquisition and image reconstruction requirements, and cannot reach the same level of hybrid imaging as our omni-tomographic approach.

US—

US imaging is arguably the most cost-effective and widely used clinical imaging modality. The role of US imaging in omni-tomography is equally important. In designs according to embodiments of this invention, the US transducer can be fixed to a physiologically relevant ROI on the patient prior to the scan. MRI compatible US systems are already commercially available. In a typical system, the US transducer and cables are shielded with aluminum foil. See Annie M. Tang, D. F. K., Edmund Y. Lam, Michael Brodsky, Ferenc A. Jolesz, Edward S. Yang Multi-modal Imaging: Simultaneous MRI and Ultrasound Imaging for Carotid Arteries Visualization, in 29th Annual International Conference of the IEEE Engineering in Medicine and Biology Society. 2007 Lyon. In addition to US Imaging, photo-acoustic imaging is an emerging modality and seems a promising direction for future integration as the technology matures.

Optical Imaging—

Optical imaging is a rapidly growing area and works by detecting molecular and cellular information via fluorescence and bioluminescence probes. Interestingly, x-rays and gamma rays can induce fluorescence and luminescence signals, which can be quite significant when fluorescence and luminescence nanoparticles (e.g. quantum dots and nano-phosphors) are in high concentrations. Due to the use of the interior imaging principle, there is unused space in the CT-SPECT ring where x-ray luminescence and x-ray fluorescence cameras could be potentially placed. Among the various possibilities, interior x-ray fluorescence CT is preferred because x-rays are not much diffusive and give strong signals with well-designed nanoparticles. As a side note, additional x-ray detectors outside the primary CT beam may be utilized for scattering tomography which depicts scattering characteristics in 3D and offers information complementary to linear attenuation characteristics, especially using spectroscopic x-ray detection.

In omni-tomography designs according to the invention, use of a slip ring technology enables data to be transmitted from various modalities while a subject is being scanned. The advantages of an omni-tomography scanner over current multimodality systems are not only valuable tomographic information within the same time window but also major space savings as well as logistic convenience.

In embodiments of the invention omni-tomography has three distinct features: (1) spatially localized in a typical scenario, (2) temporally matched as closely as possible, and (3) functionally integrated to decipher physiological complexities and dynamics. Concurrent acquisition of diversified scans of tissues will offer critical information that is unavailable in images acquired using each modality individually or using modality fusion. Each modality can be interiorized, and any two modalities can be fused. In particular, open MRI scanners are available from Fonar and other companies, giving open space between the double-donut-shaped magnets. Several companies and universities show that PET and SPECT can be placed inside an MRI aperture. It is known that components can be electromagnetically fielded. Antimagnetic materials have been reported that shield magnetic fields with superconductor-metamaterial hybrids. See Sanchez A, Navau C, Prat-Camps J, Chen D X: Antimagnets: Controlling magnetic fields with superconductor-metamaterial hybrids. New Journal of Physics 13:093034, 2011.

The omni-tomography specifications can be operably configured to target the canine model, as shown for example in FIG. 1B. The imaging field of view is 30 cm to allow space for dynamic limb and diaphragm imaging. The target ROI diameter is 5 cm. The system can produce a range of imaging indices matching the high-end clinical imaging performance or better (such as CT: 0.2 mm resolution: SPECT: ˜0.4 mm resolution; MRI: ˜1 mm; optical: ˜0.3 mm).

The omni-tomographic instrumentation can involve novel imagers and their integration: (1) an interior CT imager; (2) a stationary multi-view interior SPECT imager, (3) an interior MRI imager; and (4) an omni-tomography setup merging the first three imagers along with an optical endoscope. With the interior tomography strategy, the data acquisition speed can be much improved such as the proposed multi-view SPECT. Interior MRI is a major innovation. For example, two permanent rings can be used to synthesize a homogeneous magnetic field of ˜7 cm in diameter. Within the magnetically constant ROI, all MRI (including correction) methods can be applied after adaption. For example, the gradient field can be kept zero at an ROI slice and the gradient field varied quickly outside that level. In this way, MR data can be generated only from the ROI, since magnetic iso-regions outside the ROI are incoherently excited and negligible. A similar strategy can be used to collect MR signals from a volume of interest. For physiological studies, a remotely controllable environment (gas exchange, temperature, and various accessories) can be used within the imaging chamber, then muscular motion induced, and non-imaging signals measured.

The omni-tomographic reconstruction can be performed in a dictionary/atlas based framework. This is to revolutionize the reconstruction strategy from single-modality computation to all-modality reconstruction. An exemplary unified iterative reconstruction can proceed as follows: (1) based on the atlas an initial composite image is set; (2) an image is updated with an omni-tomographic scan; (3) a diffeomorphism is driven via flow (such as Ricci flow used to solve the Poincare conjecture) by the dictionary/atlas, yielding a Beltrami coefficient (BC) distribution; (4) the current image is refined to minimize the data discrepancy and BC-based sparsity. The key is to combine all the fidelity and penalty terms into one objective function, and minimizes it using a split-Bregman-type scheme.

The benefits of omni-tomography are numerous. For example, omni-tomography may significantly reduce costs related to oncological imaging. Typically, patients who are diagnosed with cancer will receive multiple scans of various modalities during their course of treatment. The following data from Duke University (via the website auntminnie.com) helps illustrate this point: in 2006, the average patient with lung cancer received (within two years of diagnosis) 11 radiographs, one PET scan, six CT scans, a separate nuclear medicine test, one MRI exam, two echocardiograms, and an ultrasound. It is likely that each of these scans were performed on a separate scanner within the hospital/clinic. The benefit of an omni-tomography scanner can be seen in this scenario, where a single scanner installed in a room can be used to perform most of these scans in a single visit, with periodic follow-up scans over the course of treatment. Furthermore, data collected from multiple imaging modalities on an omni-tomography scanner can be seamlessly integrated, packaged, and presented to clinicians for analysis and diagnosis. This can allow better management and tracking of the progression of the cancer.

With the current increasing trends in modality usage, it is expected that hospitals and clinics will be interested in a scanner that can support all imaging modalities, while at the same time providing more information per scan. The benefit of an omni-tomography scanner may therefore be quantified in terms of the number of image modalities per patient scan. A dual modality scanner may provide 2 image modalities/patient scan compared to an omni-tomography scanner that provides 4 to 5 imaging modalities/patient scan.

TABLE II
Components of a representative omni-tomography imaging
system (also referred to as multi-tomography) system.

| | Component | Manufacturer |
|---|---|---|
| Magnetic | Permanent | Wenzhuo |
| Resonance | magnet, | Light |
| Imaging | electronics, | Industrial |
| (MRI) | gradient/RF | |
| | coils | |
| Computed | Tube, | Varian, |
| Tomography | electronics, | Radiation |
| (CT) | and detector | Monitoring |
| | | Devices Inc. |
| Positron | Block crystal | Radiation |
| Emission | and | Monitoring |
| Tomography | coincidence | Devices Inc. |
| (PET) | circuit | |
| Single | CZT | Radiation |
| Photon | detector and | Monitoring |
| Emission | electronics | Devices Inc. |
| Computed | | |
| Tomography | | |
| (SPECT) | | |
| Ultrasound | US | Boston |
| Imaging | transducers | Scientific |
| (US) | and | |
| | electronics | |
| Optical | Near | Princeton |
| Imaging | infrared | Scientific, |
| | photon- | Amptek |
| | counting | |
| | camera, | |
| | x-ray | |
| | fluorescence | |
| | camera | |

Interior Reconstruction.

Interior tomography as a general imaging principle has been extended to other modalities, including interior electron-beam tomography (see Wang G, Yu H Y: Can interior tomography outperform lambda tomography?Proc Natl Acad Sci 107:E92-3, 2010), interior SPECT (see Yu. H Y, Yang J S, Jiang M, Wang G: Interior SPECT-exact and stable ROI reconstruction from uniformly attenuated local projections. Communications in Numerical Methods in Engineering 25:693-710, 2009; and Yang J S, Yu, H Y, Jiang M, Wang G: High order total variation minimization for interior SPECT. Inverse Problems, pending minor revision, 2011), interior MRI, and so on. Thanks to interior tomography, data acquisition module of different types can be made more compact or rather narrow to provide space and reduce cost for omni-tomography.

Interior tomography is both theoretical basis and enabling technology for omni-tomography or multi-tomography, because it or its variants (such as approximations) must be used to deal with truncated data. Representative examples to establish the feasibility of interior reconstruction in the context of omni-tomography are provided here. Note that some modalities for omni-tomography can have less truncation in the data acquisition process than others.

Interior CT.

The long-standing interior problem is to reconstruct an interior ROI only from projection data associated with lines through the ROI. Since 2007, interior tomography theory and methods have been developed to solve this interior problem in a theoretically exact and stable fashion under practical conditions. See Ye 2007; Yu 2008; Courdurier, M., et al., Solving the interior problem of computed tomography using a priori knowledge. Inverse Problems, 2008. 24: p. Article ID 065001, 27 pages (“Courdurier 2008”); and Kudo, H., et al., Tiny a priori knowledge solves the interior problem in computed tomography. Phys. Med. Biol., 2008. 53(9): p. 2207-2231 (“Kudo 2008”). In the compressive sensing (CS) framework, we proved that an interior ROI can be exactly reconstructed via the total variation (TV)/high-order TV (HOT) minimization if features to be reconstructed is piecewise constant/polynomial. See Yu I 2009; Yu II 2009; Han, W., H. Yu, and G. Wang, A total variation minimization theorem for compressed sensing based tomography. International Journal of Biomedical Imaging, 2009. 2009: p. Article ID: 125871, 3 pages (“Han 2009”): and Yang, J. S., et al., High-order total variation minimization for interior tomography. Inverse Problems, 2010. 26(3): p. 29 (“Yang 2010”). Most importantly, numerical and experimental results using interior tomography have been very encouraging.

Imaging Model—

In a fan-beam/cone-beam CT geometry, the forward and back projection operations for image reconstruction usually assume a linear integral from the detector center to the source point. Let ƒ(x) be a 2D compactly supported function. The x-ray projection is modeled by:

$$P ( a , β ) = {\int }_{0}^{∞} f ( a + t β ) d t , \qquad ( III . A .1 )$$

where aε [symbol] represents a source position, and βεS a 2D unit vector. Here, a more realistic imaging model is introduced. See Yu, H. and G. Wang, Finite Detector Based Projection Model for Super Resolution CT, in Fully3D 2011. 2011 (“Yu 2011”). Let Ωₛ⊂ [symbol] be a compact support of a source spot.

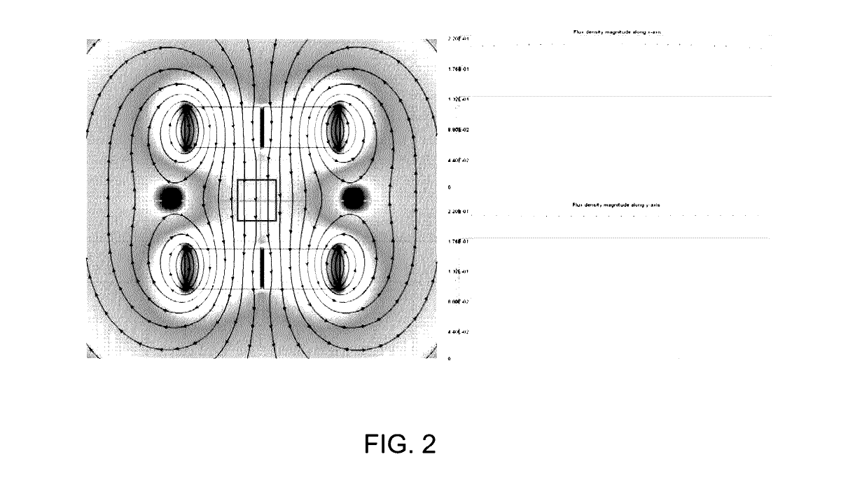

As shown in FIG. 3, the two detector elemental border points and a e, define a narrow fan-beam of angle γₐ. A unit vector from a to a point in the detector aperture is β(θ,a), θε[0,γₐ]. Assuming that the x-ray flux along any direction from aεΩₛ is I₀, the signal detected by the detector element can be computed according to Beer's law:

$$I ( a , θ ) = {I}_{0} {e}^{- {\int }_{o}^{∞} f ( n + t β ( θ , a ) ) d t} , \qquad ( III . A .2 )$$

which is equivalent to Eq. (III.B.1) after a logarithm operation ln(I₀/1(a,θ)). Considering the finite detector and source sizes, the total number of received photons becomes:

$$I = {\int }_{{Ω}_{s}} {\int }_{0}^{{γ}_{a}} I ( a , θ ) d θ d a = {I}_{0} {\int }_{{Ω}_{t}} {\int }_{0}^{{γ}_{a}} {e}^{- {\int }_{0}^{∞} f ( a + t β ( θ , s ) ) d t} d θ d a , \qquad ( III . A .3 )$$

where da represents an area differential of the focal spot. After appropriate approximation, it has been shown that Eq. (III.A.3) can be re-expressed as:

$$\overline{\overline{p}} = \frac{{\int }_{{Ω}_{s}} \frac{1}{{γ}_{a}} {\int }_{{Ω}_{b}} f ( x ) \frac{dx}{\vert \vert x - a \vert \vert} d a}{{\int }_{{Ω}_{s}} d a} , \qquad ( III . A .4 )$$

where Ω_{b} denotes the narrow fan-beam region, and dx the corresponding differential. See Yu 2011.

For digital image reconstruction, ƒ(x) is discretized as a discrete image f=(ƒ_{i,j})ε [symbol] × [symbol] , 1≦i≦N₁ and 1≦j≦Nⱼ. Define:

ƒₙ=ƒ_{i,j}, n=(i−1)×N_{J}+j, (III.A.5)

1≦n≦N and N=Nⱼ×Nⱼ, and there is vector f=[ƒ₁, ƒ₂, . . . , ƒ_{N}]^{I}ε [symbol] .

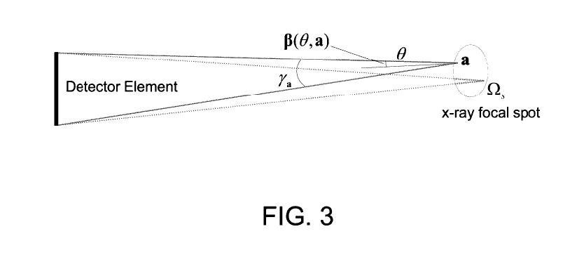

Either ƒ_{i,j} or ƒₙ can be used to represent the image. Assume that the finite focal spot Ωₛ be discretized as a_{q}, 1≦q≦Q. Let p^{q}=[p₁^{q}, p₂^{q}, . . . , p_{M}^{q}]^{I} ε [symbol] is measured data associated with the x-ray source sub-region a_{q} and all the detector elements, where M is the product of the number of projections and the number of detector elements. Eq. (III.B.4), this results in the following discrete linear system:

$$p = \frac{1}{Q} {\sum }_{q = 1}^{Q} {p}^{q} = \frac{1}{Q} {\sum }_{q = 1}^{Q} {B}^{q} f = ( \frac{1}{Q} {\sum }_{q = 1}^{Q} {B}^{q} ) f = Bf , \qquad ( III . A .6 )$$

where B^{q}=(B_{m,n}^{q})ε [symbol] × [symbol] is the linear measurement matrix for the x-ray source a_{q}. As shown in FIG. 4, the n^{th} pixel is viewed as a rectangular region with a constant value ƒₙ, the m^{th} measured datum pₘ^{q} is viewed as an integral of those areas of pixels that are partially covered by a narrow fan beam from the source sub-region a_{q} to a detector element and weighted by the corresponding x-ray linear attenuation coefficients and fan-arc lengths. Thus, the component B_{m,n}^{q} in Eq. (III.A.6) can be expressed as:

$${B}_{m , n}^{q} = \frac{{S}_{m , n}^{q}}{{L}_{m , n}^{q}} , \qquad ( III . A .7 )$$

where S_{m,n}^{q} denotes the intersection area between the n^{th} pixel and the m^{th} fan-beam path, and L_{m,n}^{q} can be approximately computed as the product of the narrow fan-beam angle γₘ^{q} and the distance from the n^{th} pixel center to the x-ray source a_{q}, which can be viewed as fan-arc length of the narrow fan-beam through the n^{th} pixel center.

Image Reconstruction—

The CS approach has recently been used in multiple biomedical imaging applications. With CS, high-quality signals and images can be reconstructed from far fewer data/measurements than required by the Nyquist sampling theory. See Donoho, D. L., Compressed sensing. IEEE Transactions on Information Theory, 2006. 52(4): p. 1289-1306; and Candes, E. J., J. Romberg, and T. Tao, Robust uncertainty principles: Exact signal reconstruction from highly incomplete frequency information. IEEE Transactions on Information Theory, 2006. 52(2): p. 489-509. The main idea of CS is that most signals are sparse (i.e. a majority of their coefficients are close or equal to zero) when analyzed in an appropriate domain. Hence, CS depends on a sparsifying transform. In CS-based image reconstruction, commonly used sparsifying transforms are: discrete gradient transforms and wavelet transforms. The discrete gradient transform was successfully utilized in CT reconstruction (see Yu I 2009; and Chen, G. H., J. Tang, and S. Leng, Prior image constrained compressed sensing (PICCS): A method to accurately reconstruct dynamic CT images from highly undersampled projection data sets. Medical Physics, 2008. 35(2): p. 660-663) given that the x-ray attenuation coefficient usually varies mildly within organs and large variations are usually found across boundary borders.

Accordingly, a CS solution to Eq. (III.A.7) can be expressed as:

$$\hat{f} = {arg m in}_{f} \vert \vert p - Bf {\vert \vert}^{2} + β TV ( f ) , \qquad ( III . A .8 )$$

where the term ∥p−Bf∥² is for the data discrepancy, TV(f) is for the image sparsity and β is a balancing parameter. The inventors have developed an iterative algorithm to solve Eq. (III.A.8.) (see Yu I 2009; and Yu, H. and G. Wang, A soft-threshold filtering approach for reconstruction from a limited number of projections. Phys Med Biol, 2010. 55(13): p. 3905-3916, “Yu 2010”) which consists of two major steps: (1) the ordered-subset simultaneous algebraic reconstruction technique (OS-SART) (see Wang, G. and M. Jiang, Ordered-Subset Simultaneous Algebraic Reconstruction Techniques (OS-SART). Journal of X-ray Science and Technology, 2004. 12(3): p. 169-177) is used to reconstruct an ROI image based on all the truncated projection data; and (2) the TV is minimized in a soft-threshold filtering framework with a pseudo-inverse discrete gradient transform (see Yu 2010). These steps are performed in an alternating manner. To accelerate the convergence, the projected gradient method is employed (see Daubechies, I., M. Fornasier, and I. Loris, Accelerated Projected Gradient Method for Linear Inverse Problems with Sparsity Constraints. Journal Of Fourier Analysis And Applications, 2008. 14(5-6): p. 764-792) to determine an optimal filtering threshold and implemented a fast iterative version (see Beck, A. and M. Teboulle, A Fast Iterative Shrinkage-Thresholding Algorithm for Linear Inverse Problems. Siam Journal on Imaging Sciences, 2009. 2(1): p. 183-202).

Numerical Simulation—

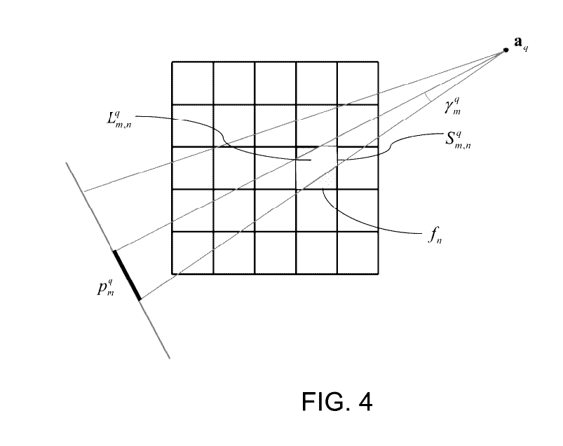

According to the preferred omni-tomography design (FIG. 1), numerical simulations were performed using a circular scanning locus of radius 45.0 cm and fan-beam geometry. An equi-space detector was placed opposite to the x-ray source with a source-to-detector distance 87.5 cm), and positioned perpendicular to the direction from the source to the system origin. The detector array consisted of 384 square elements, each of which was 0.776 mm in length, giving a total detector width of 29.7 cm. The detector size was chosen according to the commercial flat-panel detector PaxSca4030 in the 4×4 binning mode. The system was assumed to have an ideal point x-ray source, which means Q=1 in Eq. (III.A.6). This configuration can cover an interior ROI of diameter 15.1 cm. A 1000×1000 patient chest image phantom was used, covering an area of 40×40 cm². The heart was centered at the system origin. For a full-scan, 1160 equi-angular projections were acquired, which was within the range used in commercial CT scanners. According to methods of the invention, any number of projections can be acquired. FIGS. 5A-C present the interior reconstruction after 15 iterations, which shows that there is no significant difference between the interior reconstruction and the original phantom image. FIGS. 5D-E are the profiles along the horizontal and vertical white lines in FIGS. 5B-C, respectively, where the thick lines on the horizontal axis indicate the ROI.

Interior MRI.

For a preferred omni-tomography scanner, conventional MRI magnets do not seem a viable option because of their spatial bulkiness. An alternative strategy is to create a local magnetic background field (main field) such as using permanent magnets. This local magnetic field can be either homogeneous or inhomogeneous. It is possible to create a homogeneous local magnetic field using relatively small permanent magnets, as shown in FIG. 2. So long as the homogeneity of the magnetic field holds over an ROI, classical MRI approaches (and correction methods) can be easily adapted for local MRI (see a recent review paper Fessler, J. A., Model-Based Image Reconstruction for MRI, IEEE Signal Processing Magazine, 27(4): p. 81-89 and references therein, as well as our method described below to remove inhomogeneity of the local magnetic field).

Alternatively, the inventors have devised a novel approach based on an inhomogeneous local magnetic background field for local MRI, which is referred to as interior MRI. The key idea is to take advantage of surfaces with equal magnetic field strength, or “level sets”, defined by the inhomogeneous background field for spatial localization. This approach accommodates a certain degree of field non-homogeneity, and is fundamentally different from the conventional MRI which typically starts with a homogeneous main magnetic field. Thus, it reduces the size (and possibly also cost) of the main magnet and is particularly attractive for omni-tomography or multi-tomography.

Imaging Model—

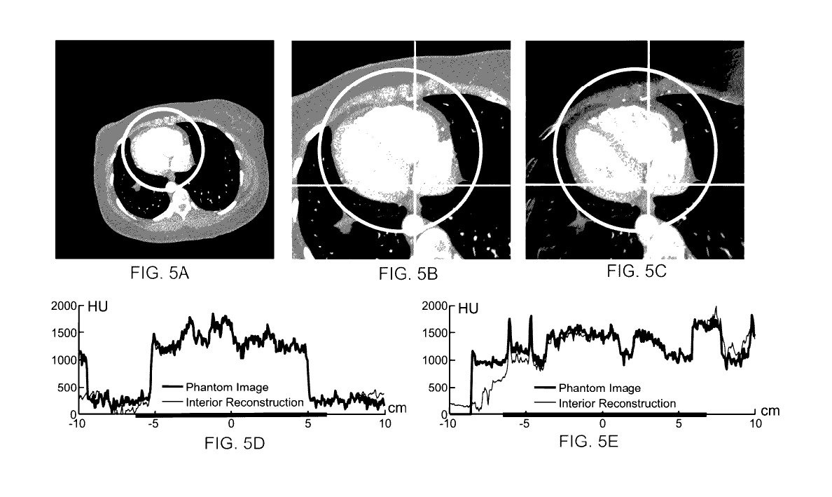

In the case of interior MRI, without loss of generality, an ROI D is represented by the union of level sets of the main field B₀ along the z-direction (in the same spirit, the main field B₀ can be dealt with whose magnetic lines are not necessarily parallel to the z-direction), i.e.,

$$D = {⋃}_{s} ( \overline{x} \text{:} {B}_{0} ( \overline{x} ) = c ) , c ∈ \{ {B}_{min} , {B}_{max} \} , \qquad ( III . B .1 )$$

where \vec{x} denotes a spatial point. In other words, the inhomogeneous magnetic field B₀ offers a natural spatial decomposition into level sets, which are constant intensity sub-regions or iso-regions. As the first step for spatial localization, the radiofrequency (RF) pulse can be tuned to excite any iso-region, as shown in FIGS. 6A-B. Then, standard x-, y- and z-linear gradient fields can be used to further localize each iso-region into desired voxels. However, this may require long data acquisition time when the number of iso-regions is large, since each iso-region needs a 3D scan. To achieve a sampling efficiency that is equivalent to that of the conventional MRI scan, a compressive sampling scheme can be used, e.g., in 2D when the iso-regions are circular.

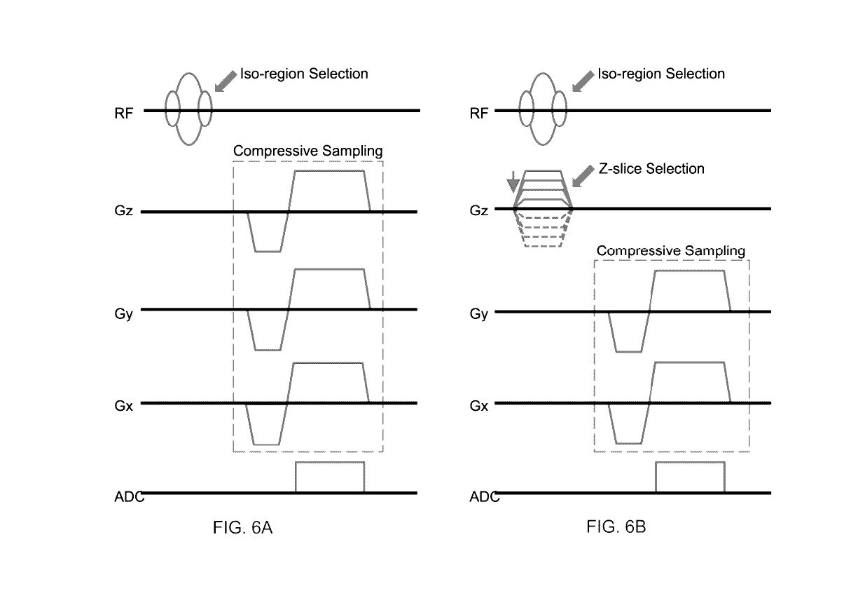

In practice, an inhomogeneous field can be well controlled. As a simple example, FIGS. 7A-C show a magnetic field consisting of cylindrical iso-surfaces with a decaying strength away from the z axis and a direction parallel to the z axis. For such an inhomogeneous magnetic field, 2D slices (e.g. transverse or z-slices) can be selected by modulating the z-gradient field appropriately. For example, a constant z-gradient field strength can be kept at one and only one z-level and vary the z-gradient field quickly outside the given z-level, as shown in FIG. 6B. Such a 2D slice intersects iso-regions and generates magnetic resonance (MR) data from the given z-slice only. In other words, by keeping the z-gradient constant at a given longitudinal location, the desirable iso-regions at the z-slice can be constructively excited, while by varying the z-gradient rapidly outside that longitudinal location, the iso-regions off the z-slice are incoherently excited for a short time, and their excitation effect can be negligible, as illustrated in FIGS. 7A-C. In this way, any 2D slice can be selected. Note that this principle can be extended to a general smooth magnetic field.

As mentioned above, in interior MRI the level sets naturally defined by an inhomogeneous main field for spatial localization can be used, and MR data can be acquired aided by standard linear gradient fields. During data acquisition, the MR signal is generated with dynamic radiofrequency (RF) pulses and can be recorded for every iso-region. For higher acquisition efficiency, the following compressive sensing scheme can be used. Without loss of generality, consider interior MRI of a thin 2D x-y slice under the main field B₀ along the z direction that is radially symmetric in the x-y plane:

B₀(r,θ,z)=B₀(r^{α}), (III.B.2)

and linear x and y gradients with the slope Gₓ and G_{y} respectively. During data acquisition, each iso-curve can be excited with the RF pulse that has the same excitation and demodulation frequency as the resonance frequency for the corresponding level set. That is, to excite the iso-curve with its radius r=r₀, the resonance frequency should be:

ω₀(r,θ)=γB₀δ(r-r₀^{α}), (III.B.3)

the demodulated signal, assuming the uniformity of the receiver coil, is proportional to:

s(t)=∫ω₀ρe^{iγ(xG}^{ₓ}^{+yG}^{_{y}}^{)t},dxdy, (III.B.4)

where ρ represents a 2D MR image to be reconstructed.

Compressive Sensing—

After the Fourier space is filled, by varying the y gradient field as the phase-encoding gradient, and acquiring the signal with the x gradient field as the frequency-encoding gradient, the iso-curve of the image at r=rₒ can be reconstructed using the inverse Fourier Transform. Continuing in this fashion, all iso-curves can be recovered and therefore the entire image can be reconstructed. This requires, however, excessive acquisition time, on the order of a 3D scan, since it takes one 2D scan for each iso-curve.

Fortunately, the inventors have devised the following data acquisition scheme which should take a similar amount of time as a conventional 2D MRI scan. Before the compressive sensing acquisition, the gradient field orientation indexes and the iso-curve indexes are randomized, respectively, and then the field orientations are sequentially paired with the iso-curves, as illustrated in FIG. 6B. According to this mutated combination, each iso-curve can be sampled under only one gradient orientation, in contrast with all the phase-encoding steps described previously.

Now, gradient fields (Gₓ, G_{y}) by (G, θ₀) can be represented as follows:

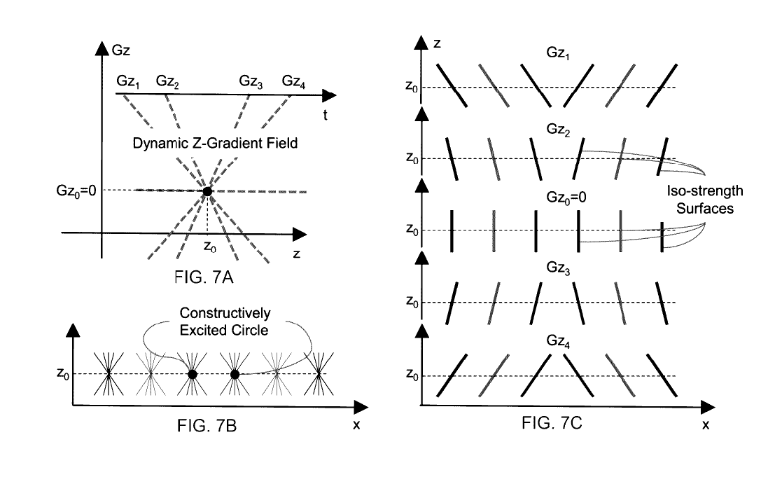

Gₓ=G cos θ_{G}, G_{y}=G sin θ_{G}, θ_{G}ε[0,π], (III.B.5)

where the orientation angle θ₀ is random but fixed for a given so-curve. The reason for randomization is as follows: for any iso-curve, since the data are acquired by varying G for some fixed angle θ₀, the spatial localization is non-unique on the circle; yet no continuous function defined on the circle can resolve the non-uniqueness. To overcome this ambiguity, start with the random selection of θ₀ for each iso-curve, and then perform image reconstruction from data associated with all the iso-curves subject to the underlying sparsity in a proper transform domain, such as the total variation (TV). With angular randomization and compressive sensing techniques, the non-unique spatial locations on the iso-curve can now be separated to satisfy certain image smoothness conditions. As a result, the entire image can be accurately reconstructed. As in FIGS. 8A-C, the image is assumed to consist of 4 iso-curves. Instead of taking all phase-encoding steps for each iso-curve, we just use one step for the iso-curve. This acquisition is random in terms of the gradient field orientation, which is from a pre-specified set of admissible angles. Then, the image can be reconstructed through a L₁-type minimization.

Image Reconstruction—

On the discrete level, the MR signal equation can be re-written in polar coordinates for the level set with r=r₀ from Eqs. (III.B.2)-(III.B.5):

$$s ( {k}_{θ} ) = {r}_{0} {B}_{0} {\vert}_{r = {r}_{0}} \int ρ ( {r}_{0} , θ ) {e}^{{ik}_{θ} [ 2 π {r}_{0} cos ( \partial - {θ}_{0} ) ]} d θ , \qquad ( III . B .6 )$$

where k_{θ} is defined as:

$${k}_{θ} = \frac{γ G}{2 π} t . \qquad ( III . B .7 )$$

Then, on the discretized Cartesian grid, an image ρ satisfying Eq. (III.B.6) can be represented by:

Aρ=s, (III.B.8)

where A is the system matrix discretized from Eq. (III.B.6) for all iso-curves, incorporating the interpolation effect from polar to Cartesian coordinates with the polar grid oversampled near the origin to maintain the integral accuracy.

Under the least-square data fidelity and the TV regularization, interior MRI can be formulated as:

$$ρ = {arg min}_{ρ} \vert \vert A ρ - s {\vert \vert}_{2}^{2} + λ \vert \vert ρ {\vert \vert}_{TV} . \qquad ( III . B .9 )$$

where λ is the regularization parameter that balances data fidelity and image smoothness. Here, the split Bregman method is applied (see Goldstein, T. and S. Osher, The split Bregman algorithm for I₁ regularized problems. SIAM J. Imaging Sci., 2009. 2: p. 323-343) as an efficient solver of Eq. (III.B.9). To demonstrate the merits of the compressive sensing technique, Eq. (III.B.9) is evaluated against the classic L2 regularization, which is similar to the Tikhonov regularization or the maximum-likelihood method:

$$ρ = {arg min}_{ρ} \vert \vert A ρ - s {\vert \vert}_{2}^{2} + λ \vert \vert ρ {\vert \vert}_{2}^{2} . \qquad ( III . B .10 )$$

Numerical Simulation—

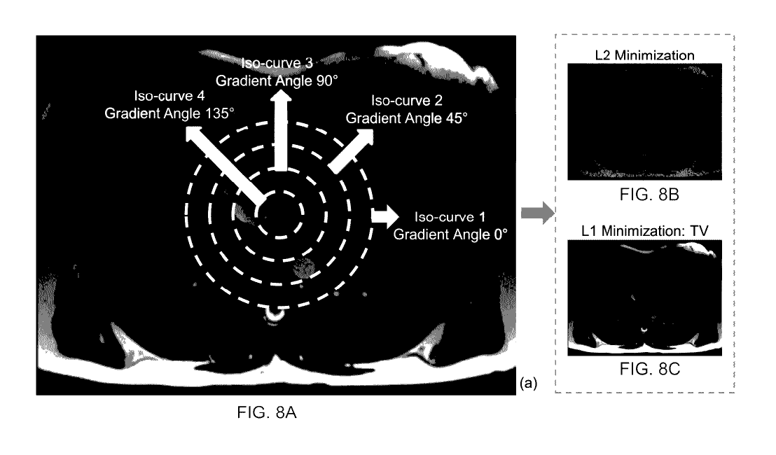

The numerical simulation was performed with an MRI cardiac image as the phantom. The L1 method was compared with the classic L2 method to demonstrate the need for compressive sensing. The results clearly indicated that the L1 method outperformed the L2 method in terms of contrast and signal to noise ratio (SNR). To further demonstrate the potential of the L1 method, the data space was under-sampled by a factor of 50%, 25%, 12.5% respectively, and the method still reconstructed well. Moreover, ROI-based reconstruction was showcased, pertinent to omni-tomography, in which MR data was only acquired within an ROI, as shown in FIG. 9. The results showed that the proposed technique enabled accurate interior image reconstruction based on a local inhomogeneous main field.

One possible drawback for the application of an inhomogeneous magnetic field is its influence on transverse relaxation time (T2*) which is, to a large degree, characterized by the B₀ non-homogeneity. This can be mitigated by reducing the strength of the measurement field and using a proper sequence such as spin-echo. New imaging techniques such as UTE and SWIFT (see Robson, M. D., et al., Magnetic Resonance: An Introduction to Ultrashort TE (UTE) Imaging. Journal of Computer Assisted Tomography, 2003. 27(6): p. 825-846; and Idiyatullin, D., et al., Fast and quiet MRI using a swept radiofrequency. Journal of Magnetic Resonance, 2006. 181(2): p. 342-349) that image objects consisting of spins with extremely short T2″ can be adapted for this case, with negligible time between excitation and signal acquisition.

The field of MRI has accumulated a huge amount of results, most of which are based on a homogeneous magnetic background field. It is expected that the idea on MRI based on an inhomogeneous magnetic background field will bring new results that can be practically used in the near future.

The inhomogeneous magnetic field based MR model, compressive sampling and reconstruction techniques for interior MRI can be extended to other types of 2D and 3D magnetic field level sets and other MR sequences. In particular, to accelerate the data acquisition process, multiple level sets can be simultaneously excited with RF pulses of different frequencies, and multi-frequency signals can be received through multi-channels. In the inhomogeneous magnetic background, we can combine MRI and the emerging magnetic particle imaging (MPI) to extract even more information at the functional, cellular and molecular levels.

Interior SPECT.

Inspired by the development of interior tomography for CT, interior SPECT techniques were also developed assuming a constant attenuation background (further work in progress). SPECT is used to reconstruct a radionuclide (radiotracer) source distribution from externally measured gamma ray photons. A gamma camera is used to acquire multiple 1D/2D projections from various angles. Then, a CT-like algorithm is applied for image reconstruction. Based on interior tomography results (see Ye 2007; Yu 2008; Courdurier 2008; and Kudo 2008), it was shown by the inventors in 2008 that theoretically exact interior SPECT is feasible from uniformly attenuated local projection data, aided by prior knowledge of a sub-region in the ROI. See Yu, H. Y., et al., Interior SPECT-exact and stable ROI reconstruction from uniformly attenuated local projections. Communications in Numerical Methods in Engineering, 2009. 25(6): p. 693-710. With CS techniques (Han 2009; Yang 2010; Yu I 2009; and Yu II 2009), it was further proven by the inventors that if an ROI is piecewise polynomial, then it can be uniquely reconstructed from truncated SPECT data directly through the ROI. See Yang, J., et al., High Order Total Variation Minimization for Interior SPECT. Inverse Problems, 2011: p. Pending Revision (“Yang 2011”); published http://www.imaging.sbes.vt.edu/BIDLib/CT/YangYuJiangWang_HighOrderTVminSPECT_PMID22215932.pdf.

Imaging Model—

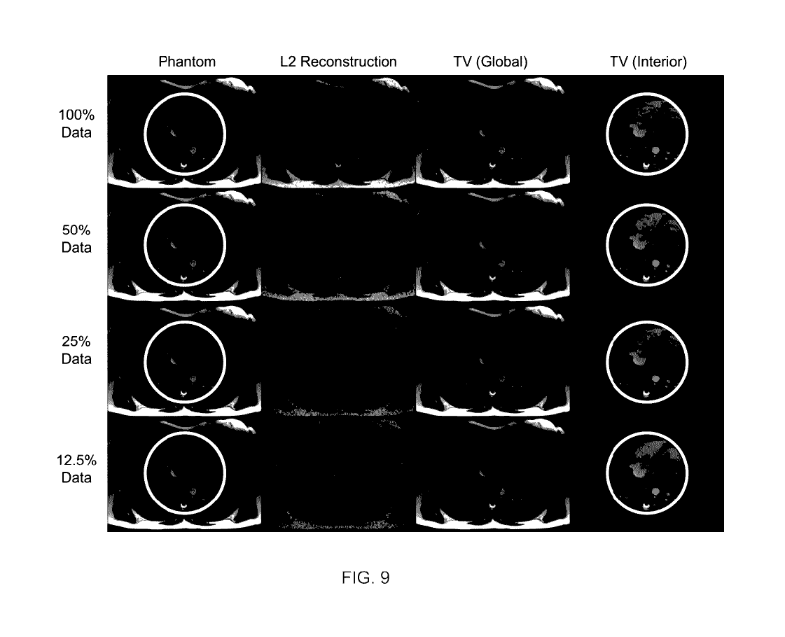

Let ƒ(x) be a 2D smooth distribution function on a compact support Ω with x=(x,y)εΩ. In a parallel-beam geometry illustrated in FIG. 10, SPECT projections of ƒ(x) can be modeled as an attenuated Radon transform (see Rullgard. H., An explicit inversion formula for the exponential Radon transform using data from 180˜. Ark. Mat., 2004. 42: p. 353-362

$${P}_{o} ( θ , s ) = {\int }_{- ∞}^{∞} f ( s θ + t {θ}^{⊥} ) {e}^{- {\int }_{t}^{∞} μ ( s θ + t {θ}^{⊥} ) d t} d t , \qquad ( III . C .1 )$$

where the subscript “o” denotes original projection data, θ=(cos θ, sin(θ)), θ^{⊥}=(−sin θ, cos(θ)), and μ(x) the attenuation coefficient map on the whole support. In practice, the attenuation map can be approximated as a uniform distribution

$$μ ( x ) = \{ \begin{matrix}{μ}_{0} & x ∈ Ω \\ 0 & x ∉ Ω\end{matrix} , \qquad ( III . C .2 )$$

where μ₀ is a constant. Since the object function is compactly supported, the length of the intersection between the support Ω and the integral line for Pₒ(θ,s) can be determined. Without loss of generality, this length is denoted as tₘₐₓ(θ,s), and Eq. (III.C.1) become

$${P}_{o} ( θ , s ) = {e}^{- {μ}_{q} {t}_{max} ( θ , s )} {\int }_{- ∞}^{∞} f ( s θ + t {θ}^{⊥} ) {e}^{{μ}_{0} t} d t . \qquad ( III . C .3 )$$

Assume that the compact support Ω and the constant coefficient μ₀ are known. By multiplying a weighting factor e^{μ}^{₀}^{f}^{ₘₐₓ}^{(θ,s)}, the projection model for SPECT is reduced to

$${P}_{w} ( θ , s ) = {P}_{o} ( θ , s ) {e}^{{μ}_{0} {t}_{max} ( θ , s )} = {\int }_{- ∞}^{∞} f ( s θ + t {θ}^{⊥} ) {e}^{{μ}_{0} t} d t , \qquad ( III . C .4 )$$

where the subscript “w” indicates weighted projection data. In this context, CT may be regarded as a special case of SPECT. However, generally CT reconstruction techniques cannot be directly used for SPECT.

The discrete CT model Eq. (III.A.6) with Q=1 can be extended to the SPECT case:

P_{w}=\tilde{B}f, (III.C.5)

where \tilde{B}=(B_{m,n}w_{m,n}^{μ}^{ₒ}), and w_{m,n}^{μ}^{ₒ} is the corresponding discrete term e^{μ}^{ₒ}^{t} in Eq. (III.C.4).

Image Reconstruction—

Suppose that ƒ(x) is a piecewise n-th (n≧1) order polynomial function in Ω (see Yang 2011): that is, Ω can be decomposed into finitely many sub-domains {Ωᵢ}_{fα}^{I}^{ᵣ} such that:

ƒ(x)=ƒᵢ(x), for xεΩᵢ, 1≦i≦Iᵣ, (III.C.6)

where ƒᵢ(x) is a n-th order polynomial function, and each sub-domain Ωᵢ is adjacent to its neighboring sub-domains Ωⱼ with piecewise smooth boundaries Γ_{i,j}, jεNᵢ. Then, the high-order total variation can be defined as (Yang 2011)

$${HOT}_{n + 1} ( f ) = {\sum }_{i = 1}^{{t}_{c}} {\sum }_{j > i , j ∈ {N}_{i}} {\int }_{{Γ}_{i , j}} \vert {f}_{i} - {f}_{j} \vert d s + {\int }_{Ω} min \{ \sqrt{{\sum }_{i = 0}^{n + 1} {( \frac{{\partial }^{b + 1} f}{\partial {x}^{n} \partial {y}^{n + 1 - r}} )}^{2}} , \sqrt{{( \frac{\partial f}{\partial x} )}^{2} + {( \frac{\partial f}{\partial y} )}^{2}} \} d x \qquad ( III . C .7 )$$

where the second term is a Lebesgue integral. Here, the following approximation is used (Yang 2011):

$${HOT}_{2}^{dis} = {\sum }_{u , v} \sqrt{{( {D}_{11} ( u , v ) )}^{2} + {( {D}_{12} ( u , v ) )}^{2} + {( {D}_{22} ( u , v ) )}^{2}} , \qquad ( III . C .8 )$$

where D₁₁(u,v)=ƒ_{u+1,v}+ƒ_{u-1,v}−2ƒ_{N,v}, D₁₂(u,v)=(ƒ_{u+1,v+1}+ƒ_{u-1,v-1}−ƒ_{u-1,v+1}−ƒ_{u+1,v-1})/4, and D₂₂(u,v)=ƒ_{u,v+1}+ƒ_{u,v-1}−2ƒ_{u,v} are the second-order finite differences.

With HOT₂, the solution of interior SPECT can be found as:

$$\hat{f} = {arg max}_{f} \vert \vert {P}_{w} - \tilde{B} f {\vert \vert}^{2} + β {HOT}_{2} ( f ) . \qquad ( III . C .9 )$$

Comparing Eqs. (III.C.9) with (III.A.8), the same computational structure is shared. Therefore, an interior SPECT algorithm based on the interior CT algorithm was implemented (Yang 2011). The major difference between the two algorithms is computation of the steepest gradient direction.

Numerical Simulation—

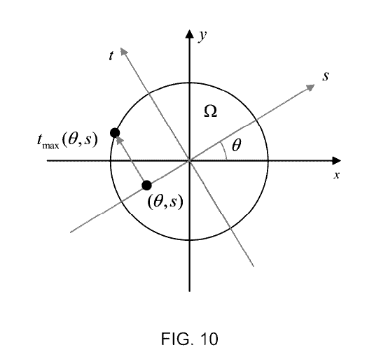

As shown in FIGS. 11A-F, a SPECT cardiac perfusion image was downloaded from the Internet and modified into a realistic 128×128 image phantom, covering an area of 128×128 mm². It represents a radionuclide distribution in a human heart. In this simulation, a constant attenuating background μ₀=0.15 cm⁻¹ on a compact support of a standard patient size was assumed. An equi-spatial detector array consisting of 78 detector elements, each of which was 1.0 mm in length was used. For a full-scan, 128 equi-angular projections were acquired. Clearly, the realistic image phantom does not satisfy the piecewise polynomial model in the case of n=11. To improve the stability of interior SPECT, two additional constraints were incorporated into the OS-SART loop in the projection-onto-convex-sets (POCS) framework: (1) non-negativity, which means that the radionuclide distribution should not be negative, therefore negative values were made zero during the iterative process; (2) compactness, which means that the radionuclide distribution should be inside the human body, and therefore the pixels outside a specified body-contour were iteratively made zero. It can be seen from FIGS. 11A-F that interior SPECT produced excellent results, even if the piecewise polynomial model is not exactly satisfied.

Interior X-Ray Fluorescence CT.

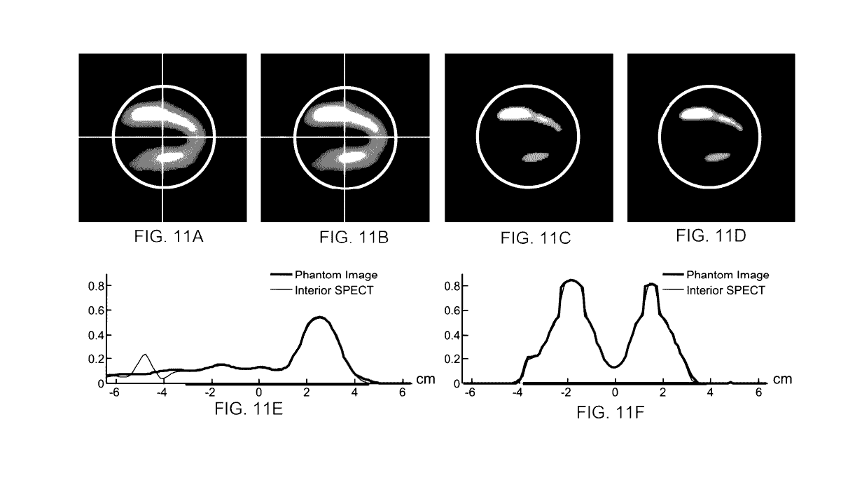

X-ray fluorescence techniques are widely used for elemental analysis (see Cesareo, R.a.S.M., A New Tomographic Device Based on the Detection of Fluorescent X-Rays. Nuclear Instruments & Methods in Physics Research Section a-Accelerators Spectrometers Detectors and Associated Equipment, 1989. 277(2-3): p. 669-672 (“Cesareo 1989”), and offer excellent sensitivity to trace elements down to the picogram level. Nanoparticles are particularly useful as imaging probes because of their unique and non-toxic physicochemical properties (see Minchin, R.F.a.D.J.M., Minireview Nanoparticles for Molecular Imaging—An Overview. Endocrinology, 2010. 151(2): p. 474-481). For example, nanoparticles can leak into the tumor by way of blood circulation and accumulate within it. X-ray fluorescence computed tomography (XFCT) can be used for 3D mapping of nanoparticles inside a human or animal subject. As shown in FIG. 12, most XFCT studies use a pencil-beam of x-rays to illuminate an object and cause emission of fluorescent x-rays from a variety of elements. The fluorescent x-rays are collected by a sensitive x-ray spectrometer, and can be used to identify various elements of interest. Also, the object is scanned and rotated in the first-generation CT geometry to obtain sufficient data for reconstruction of the elemental distributions (see Cesareo 1989).

Imaging Model—

When an x-ray beam travels through a tissue, it undergoes photoelectric interactions with inner shell electrons of atoms. The x-ray intensity distribution on the primary path can be computed by Beer's law:

$$f ( α , s , t ) = {I}_{0} exp ( - {\int }_{- ∞}^{∞} {μ}_{t} ( s , {t}^{′} ) d {t}^{′} ) \qquad ( III . D .1 )$$

where Iₒ is the x-ray source intensity, μₜ the attenuation coefficient which can be obtained from CT, and (s,t) are rotation coordinates of (x,y) s=x cos α+y sin α, r=−x sin α+y cos α. When x-ray photons interact with matter, the involved atoms isotropically emit characteristic x-rays with intensity proportional to the product of the absorbed x-ray flux rate and the fluorescent x-ray yield at a point (see T. Yuasa, M. A., T. Takeda, M. Kazama, A. Hoshino, Y. Watanabe, K. Hyodo, F. A. Dilmanian, T. Akatsuka, and Y. Itai, Reconstruction method for fluorescent x-ray computed tomography by least-squares method using singular value decomposition. IEEE Trans. Nucl. SCI., 1997.44: p. 54-62):

$$g ( α , s , t ) = {ɛμ}_{ph} f ( α , s , t ) {\int }_{{θ}_{1}}^{{θ}_{2}} exp [ - {\int }_{0}^{∞} {μ}_{F} ( s - b sin β , s + b cos β ) d b ] d β \qquad ( III . D .2 )$$

where μₚₕ is the photoelectric linear attenuation coefficient, ε the fluorescent x-ray yield, θ₁ and θ₂ define the angular aperture of the x-ray fluorescence detector, as shown in FIG. 12. The total flux rate of the fluorescent x-ray reaching the fluorescence detector is obtained by integration

$$I ( α , s ) = {\int }_{- ∞}^{∞} f ( α , s , t ) g ( α , s , t ) ρ ( s , t ) d {t}^{*} \qquad ( III . D .3 )$$

where ρ(s,t) is the concentration of nanoparticles.

Image Reconstruction—

XFCT reconstruction is a linear inverse problem when the linear attenuation coefficient distributions of the object are known at the energies of the incident and fluorescent x-rays. Hence, Eq. (III.D.3) can be discretized into the matrix format using numerical integration methods:

AX=B, (III.D.4)

where X is an discretized image in terms of a nanoparticle density distribution, B represents measured fluorescent signals, and A the system matrix from Eq. (III.D.3). Using the CS principle, we incorporated the L1 norm regularization for the sparse representation of the nanoparticle density distribution subject to the data constraint. Finally, the solution can be obtained as follows:

$$\hat{X} = {arg min}_{x} \{ \vert \vert AX - B {\vert \vert}^{2} + λ \vert \vert TX {\vert \vert}_{1} \} \qquad ( III . D .5 )$$

where T represents a sparse transform, and λ a regularization parameter. Eq. (III.D.5) can be efficiently solved using a contemporary optimization method such as the Bregman iteration scheme. See Teboulle, A.B.a.M., A Fast Iterative Shrinkage-Thresholding Algorithm for Linear Inverse Problems. Slam Journal on Imaging Sciences, 2009. 2: p. 183-202 (“Teboulle 2009”).

Numerical Simulation—

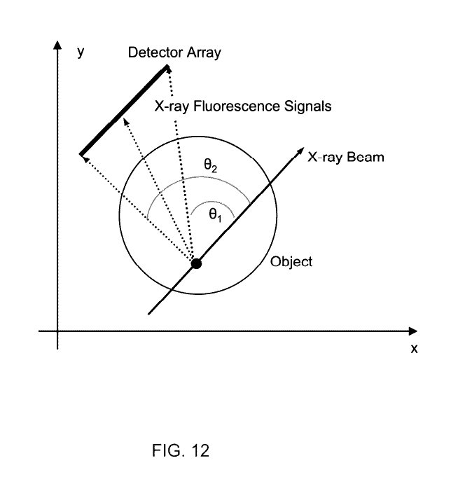

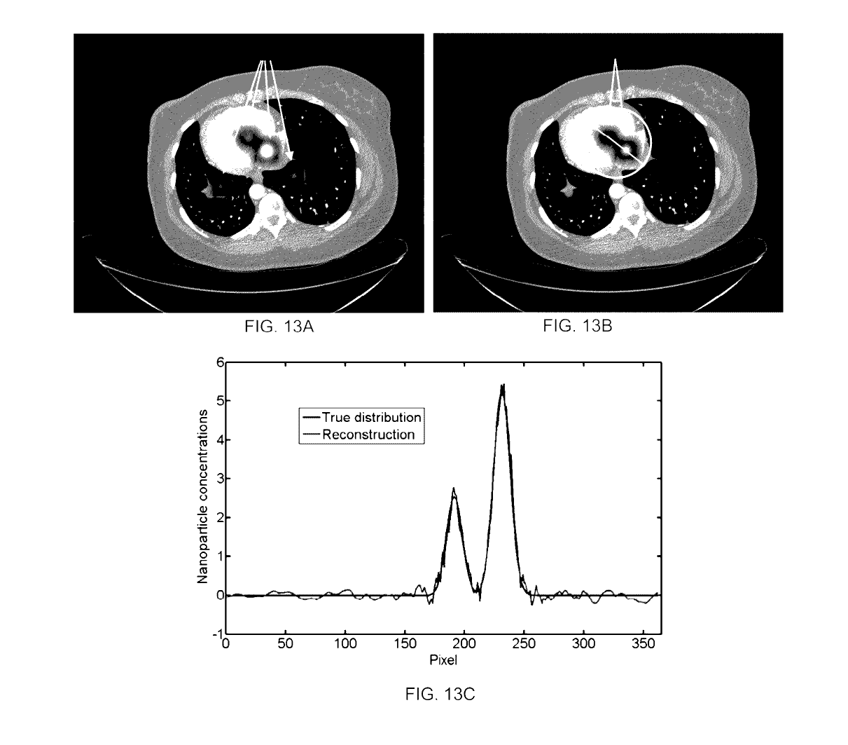

A numerical test of interior x-ray fluorescence computed tomography for a 2D patient slice was performed. The phantom consisted of gold nanoparticles accumulated in four circular regions with radii of 3.0 mm, 7.0 mm, 4 mm and 5 mm and Gaussian-like concentration distributions of 3 μg/ml, 5 μg/ml, 3.5 μg/ml, and 4 μg/ml, respectively. Two additional clusters of nanoparticles were targeted in an ROI. An x-ray source of 110 keV and 30 mA was collimated into a pencil beam to irradiate the phantom. The image acquisition was repeated 100 times when the x-ray pencil beam was translated in a 0.1 mm increment for a given view to cover the ROI. The parallel-beam imaging geometry was rotated 50 times to span a 180° range evenly around the ROI, The projection data were generated according to Eq. (III.D.3), and Poisson noise was added to simulate realistic experimental conditions. The image was reconstructed using the shrinkage-thresholding algorithm based on Eq. (III.D.5). See Teboulle 2009. FIGS. 13 A-C present a typical interior XFCT reconstruction, which is in excellent agreement with the true image.

Grand Fusion in Three Aspects.

Being consistent with the omni-tomography or multi-tomography concept, the inventors have conceived three inter-related lines of grand fusion: first, an architectural fusion that merges all or many imaging modalities into a single gantry; second, a component fusion that packs all or many detectors into a single device or chip; and third, a methodical fusion for data processing and image reconstruction in a unified framework. As previous sections of this specification discussed the architectural effort, here the others are discussed.

As far as the component fusion is concerned, there are already various detector systems that are designed on a chip. A micro-electromechanical-device (MEMS) US transducer has been built and characterized. See John J. Neumann, D.W.G.a.I.J.O. Comparison of piezoresistive and capacitive ultrasonic transducers. 2004. The piezoresistive and capacitive transducer types have been compared, showing that the piezoresistive transducer is superior to its capacitive counterpart. As for MRI, MRI on a chip has been explored (see Hassibi, A., A. Babakhani, and A. Hajimiri, A Spectral-Scanning Nuclear Magnetic Resonance imaging (MRI) Transceiver. Solid-State Circuits, IEEE Journal of, 2009. 44(6): p. 1805-1813), and a wireless MRI transmitter is feasible. See Rofougaran, A., et al., A single-chip 900-MHz spread-spectrum wireless transceiver in 1-&mu;m CMOS. I. Architecture and transmitter design. Solid-State Circuits, IEEE Journal of, 1998. 33(4): p. 515-534. In addition to solid-state detectors for SPECT and CT, a CZT detector is also feasible for PET. This is made possible with special readout chips, such as the RENA3 IC chip. Also, digital systems are available for SPECT with CZT detectors and PET with crystal systems and avalanche photodiodes. The primary merit of a single detector chip for multimodality imaging is simultaneous data acquisition. The bulkiness is not attractive for the detectors that are based on scintillation crystals (PET/SPECT), transducers (US), or magnet coils (MRI), when available space is an issue. Using the multi-component technology, omni-tomographic imaging would be easier to implement. With a sufficiently large number of omni-tomographic imaging chains together, ultrafast or even instantaneous omni-tomographic imaging can be achieved. This point was touched upon in our previously proposed multi-source interior tomography project. See Wang, G., H. Yu, and Y. Ye, A scheme for multi-source interior tomography. Med Phys, 2009. 36(8): p. 3575-3581. A second benefit of the component-level integration is cost reduction via mass production. Easier system maintenance is another potential benefit.

Lastly, the methodology fusion can be a very exciting topic. Although various modalities use different physical mechanisms, the imaging target is the same human or animal subject. As such, domain knowledge on this subject should be maximally utilized such as in the form of a population-based statistically-driven elastic atlas. In this regard, excellent work in the area of post-hoc image registration can be adapted for omni-tomographic reconstruction. See Kok, P., et al., Articulated planar reformation for change visualization in small animal imaging. IEEE Trans Vis Comput Graph, 2010. 16(6): p. 1396-404; Khmelinskii, A., et al., Articulated Whole-Body Atlases for Small Animal Image Analysis: Construction and Applications. Mol Imaging Biol, 2010; and Baiker, M., et al., Atlas-based whole-body segmentation of mice from low-contrast Micro-CT data. Med Image Anal, 2010. 14(6): p. 723-37.

Then, constraints can be applied for correlation among information from component modalities and consistence with our prior knowledge. The newly developed CS-framework can be used to unify the data process and image reconstruction. See Wang G. G. H., Zhang J, Weir V, Xu X C, Cong A, Bennett J, Medical Imaging, in Biomedical Engineering Education and Advanced Bioengineering Learning: Interdisciplinary Concept, Z. O. Abu-Faraj, Editor, in press, IGI Global: Hershey. Pa.; Osher, S., et al., An Iterative Regularization Method for Total Variation-Based Image Restoration. Multiscale Modeling & Simulation, 2005. 4(2): p. 460-489; Osher, S. and J.A. Sethian, Fronts propagating with curvature-dependent speed: Algorithms based on Hamilton-Jacobi formulations. Journal of Computational Physics, 1988. 79(1): p. 12-49; and Osher. S.J.a.F., R., Level Set Methods and Dynamic Implicit Surfaces, in Applied Mathematical Science. 2002. Springer.

Enhancement with New Modalities.

New medical imaging modalities are emerging, making omni-tomography both increasingly powerful and challenging. To meet this challenge, it appears that some form of interior imaging is unavoidable for simultaneous tomographic reconstructions using an increasing number of modalities. The power of omni-tomography lies with the increased relevance of in vivo information collected over an ROI in a single, simultaneous scan.

As mentioned above, x-ray CT is moving towards multi-energy imaging. The enabling technology is spectroscopic (energy-sensitive) detectors. For example, Medipix is a series of spectroscopic detectors for x-ray micro-imaging, developed in collaboration with the European Organization for Nuclear Research (CERN). There are three current generations of this detector: Medipix1 has detector cells each measuring 170×170 μm²; Medipix2 refines that to 55×55 μm², yet the performance is limited by the charge cloud effect: Medipix3 will have special circuitry to allow charge deposition analysis without spectral distortion, and support eight energy thresholds.

Recently, Pfeiffer et al. have made major progress towards x-ray phase-contrast and dark-field imaging with a conventional (non-coherent) x-ray tube. See Pfeiffer. F., et al., X-ray dark-field and phase-contrast imaging using a grating interferometer. Journal of Applied Physics, 2009. 105(10): p. 102006-102006-4. The key idea uses phase stepping interferometry based on the Talbot effect, a periodic self-imaging phenomenon, to extract phase shift and small-angle scattering information. This approach relies on the use of three gratings to: define small imaging apertures and produce individually coherent secondary sources from a hospital-grade x-ray tube; constructively superimpose interference fringes at an appropriate distance; detect signals via a correlation process from wave-fronts distorted by an object; and form projective images from which tomography is feasible.

Although in this specification is presented only one case study on interior reconstruction per selected imaging modality, some modalities have multiple variants to extract distinct yet complementary information. For example, MRI supports many imaging modes, including: dynamic contrast-enhanced MRI, diffusion-weighted MRI, functional MRI, pharmacologic MRI, MR elastography, MR temperature mapping, and MR spectroscopy, and more.

Recently, magnetic particle imaging (MPI) was developed as a complementary mode to MRI. MPI detects ferromagnetic nanoparticles when they are in field-free regions within a static magnetic field. The harmonic signals are induced by oscillating fields that only influence nanoparticles in field-free regions. The conventional MPI design may support MRI, if interior imaging techniques are developed assuming a non-homogeneous magnetic field. The single-sided MPI design can be a candidate for omni-tomography in the preclinical setting.

In addition to those discussed above, there are certainly other potential modalities that could be integrated with omni-tomography, such as microwave tomography and endoscopic imaging. Furthermore, novel contrast agents and nanoparticles can be combined with omni-tomography to deliver critical information. See Jin, Y., et al., Multifunctional nanoparticles as coupled contrast agents. Nat Commun, 2010. 1: p. 41. It is the ultimate goal of omni-tomography that all these imaging modes can be nonexcusively performed at the same time so that we can gain insight comprehensively, complementarily, and concurrently.

Key to Systems Biology.

With respect to the IUPS Physiome Project, omni-tomography, or multi-tomography, offers the best opportunities to observe well-registered tempo-spatial features in an unprecedented fashion and may reveal many unknown physiological, pathological, pharmaceutical and interventional interactions in vivo, and significantly improve the sensitivity and specificity of basic research, diagnosis and monitoring. Imaging technology development must target important biomedical problems. For example, biotechnology and bioinformatics have been developed to decode:

(1) Genomic/epigenetic signals at the DNA level associated with various forms of genomic signatures (e.g. single mutations, rare mutations, SNPs, copy number changes, indels, genomic instability index, etc.);

(2) Gene expressions at mRNA/miRNA/shRNA/gene/exon/splicing levels, and their various functions;

(3) Protein expression (protein complex, metabolic, etc.);

(4) Complex interactions and networks among these players on multi-scales.

Many of these studies are currently limited to cells (tissue samples, cell lines, etc.) and rarely go beyond in vitro studies, yet in vivo imaging should help facilitate translation to the organ/system/body level, where these advances should be applied. In particular, there are several unique and critical features that biomedical imaging should provide: (a) in vivo: monitoring cells in their native tissue environment for the most accurate picture of their true behaviors; (b) tomographic: 3D mappings on multi-scales from cells, tissue, organs, systems, all the way to the whole body; (c) dynamic: temporal evolution of the system within broad ranges of time windows; (d) comprehensive: biological system interactions are extremely complicated and time-sensitive, thus each imaging modality should contribute some important facet about the system in a temporally-sensitive fashion. The combination of these four features is our sense of omni-tomography and its uniqueness for biomedical applications.

Systems biology should be a great driver for omni-tomography. Consider, for example, a biological subject as a system with inputs, circuitry with feedback loops, and outputs. An omni-tomographic imaging system according to the invention, with significant help from imaging probes, can image some components of such a system in multiple dimensions (e.g. time, space, general characteristic dimensionalities). One can imagine the numerous ways this imaging system would be attractive and even indispensible to researchers. Perhaps, an omni-tomographic imaging platform to life scientists will be like the Large Hadron Collider to particle physics. In the following two subsections, let us examine some specific potential applications of omni-tomography.

Insight into Major Diseases.

Cardiovascular disease is the leading cause of death globally. The annual incidence of acute coronary syndrome (ACS) for individuals in Europe is estimated to be 1:80 to 1:170, while the incidence of chest pain leading to hospital admission for suspected ACS is much higher. Catheterized fluoroscopic angiography has been the original clinical standard for identifying stenotic lesions in the coronary arteries. Yet, other diagnostic imaging techniques have been researched and applied for this purpose, such as CT, MRI, PET, SPECT, intra-coronary ultrasound, and optical coherence tomography. The latest research emphasis is to improve understanding of pathobiology and genetics behind coronary artery diseases. There are critical and immediate needs for significantly better diagnostic performance despite the impressive advancement of imaging technology. For example, the ability to model high risk atherosclerotic lesions would be clinically invaluable. The goal would be to develop imaging methods and computational models to identify and predict high-risk lesions that may rupture, leading to coronary thrombosis and myocardial infarction. If our proposed system could be used to study preclinical infarction models, predict various outcomes, and promote clinical translations, then it would be a tremendous tool for cardiovascular imaging.

Cancer is also a major category of highly complicated and fatal diseases. Roughly speaking, cancer causes over 10% of all human deaths worldwide, and the rate is rising in the developing world. Diagnosis of cancer requires in vivo and in vitro imaging examination, but is often discovered too late for some patients. The prognosis would be better if early screening was sensitive and specific. The treatment will be more effective if it could be guided by powerful imaging and sensing techniques, preferably with tumors clearly labeled. Oncological imaging already uses al the anatomical, functional, cellular and molecular modalities. For example, lung cancer imaging has utilized all major medical tomography modalities. CT defines air-tissue interfaces, detects nodules and tumors, and provides quantitatively accurate information. MRI measures airways and function with hyperpolarized helium-3. PET and SPECT improve lung cancer diagnosis and staging with radiotracers. Omni-tomography promises to be an ideal cancer imaging tool. It could have the ability to quantify malignancy, without the need for extra invasive procedures like biopsies. Also, a futuristic feature might use the omni-tomography data to build models and create a knowledge depository linked to genetic/epigenetic information and patient histories. Furthermore, such a depository could be leveraged with information from drug databases, human genomic databases, cellular structure and functional databases, and more. Ultimately, with all this quantitative information at hand, we could envision personalized medicine where an individuals' physiology and morphology data, obtained from a single scan in an omni-tomography system, could be algorithmically compared with a database to design a pharmaceutical therapy uniquely effective for that individual's biological makeup.

Platform for Drug Development.

The inventive omni-tomographic system may be first developed for drug development and testing in animal models, with the aid of multimodality probes. Engineering imaging probes visible to multiple modalities is a hot topic in the field of bionanotechnology, and will have a profound impact on drug development and molecular medicine. Recently, exciting work has been reported on a combination of multiple nano-millimeter-sized components to facilitate multimodality imaging and even enable new imaging modes, such as with iron oxide and gold-coupled core-shell nanoparticles (NPs) with well-defined characteristics (shell thickness, core-shell separation, electronic, magnetic, optical, thermal, and acoustic).

There is a huge potential impact of combining the inventive omni-tomography system with multimodality probes, and especially in the field of pharmaceutical research. In this context, pharmacological interactions and dynamics can be noninvasively investigated from various aspects with a singular platform and probe. Furthermore, this combination could enable efficient repetition in longitudinal studies of animal models for these purposes. For example, preclinical pharmaceutical research could be the first area to employ omni-tomography, as these studies are typically not subject to lengthy restrictive FDA approvals, and will set a stage for subsequent translational research and clinical trials. It is expected that omni-tomography, given its depth, width, locality and flexibility, will outperform the current successes with modality fusion.

The present invention has been described with reference to particular embodiments having various features. It will be apparent to those skilled in the art that various modifications and variations can be made in the practice of the present invention without departing from the scope or spirit of the invention. One skilled in the art will recognize that these features may be used singularly or in any combination based on the requirements and specifications of a given application or design. Other embodiments of the invention will be apparent to those skilled in the art from consideration of the specification and practice of the invention. Where a range of values is provided in this specification, each value between the upper and lower limits of that range is also specifically disclosed. The upper and lower limits of these smaller ranges may independently be included or excluded in the range as well. As used in this specification, the singular forms “a,” “an,” and “the” include plural referents unless the context clearly dictates otherwise. It is intended that the specification and examples be considered as exemplary in nature and that variations that do not depart from the essence of the invention are intended to be within the scope of the invention. Further, the references cited in this disclosure provide general background about the specific imaging modalities that can be incorporated into omni-tomography systems according to the invention. Although only some of the modalities and underlying technology incorporated into those modalities is discussed in detail herein, all of the references cited are relied on for purposes of providing a detailed disclosure of the invention and as such all cited references are incorporated by reference herein in their entireties.

1. A patient imaging system comprising:
a gantry having a plurality of imaging modalities;
said imaging modalities operably configured for simultaneous or concurrent signal acquisition to create at least one dataset for performing reconstruction of a region-of-interest of the patient, said region-of-interest comprising a region less than a full transaxial slice of the patient; and
at least one processor operably configured to process said at least one dataset to generate an image representation of said region-of-interest of the patient.

2. The imaging system of claim 1 wherein said imaging modalities are CT and SPECT.

3. The imaging system of claim 1 wherein said imaging modalities are CT and MRI, said MRI imaging modality has a homogeneous or inhomogeneous magnetic background field only over said region-of-interest.

4. The imaging system of claim 2 wherein a plurality of modalities are fused to image said region-of-interest.

5. The imaging system of claim 2 wherein said reconstruction is performed in a compressed sensing framework.

6. The imaging system of claim 2 wherein said reconstruction is an all-modality reconstruction from datasets collected by all involved modalities.

7. The imaging system of claim 2 wherein images of said region-of-interest of the patient are obtained in a single, simultaneous scan.

8. The imaging system of claim 2 wherein images of said region-of-interest of the patient are obtained from intermingled scans that are completed within a time period that is shorter than the total time needed to perform each scan separately.

9. The imaging system of claim 2 wherein said gantry is comprised of at least one ring.

10. The imaging system of claim 2 wherein said at least one gantry is comprised of at least one partial ring.

11. The imaging system of claim 2 wherein said CT and SPECT imaging modalities are located on an inner ring and a PET imaging modality located on an outer ring.

12. A system for imaging a region-of-interest of a patient comprising:
an MRI imaging modality having a homogeneous or inhomogeneous magnetic background field only over said region-of-interest;
said MRI imaging modality operably configured to obtain a dataset of said region-of-interest, said region-of-interest comprising a region less than a full transaxial slice of the patient; and
a processor operably configured to process said dataset to generate an image representation of said region-of-interest of the patient.

13. The imaging system of claim 12 wherein said magnetic background field is modulated by a gradient field to form sub-regions each of which generate magnetic signals from said region-of-interest while other signals from regions outside said region-of-interest will be randomized and suppressed.

14. The imaging system of claim 12 wherein said system performs compressive sensing based reconstruction from data relating to said region of interest.

15. The imaging system of claim 12 further including a CT imaging modality.

16. The imaging system of claim 15 further including a PET or SPECT imaging modality.

17. The imaging system of claim 14 which can be aided by information from another imaging modality such as CT.

18. The imaging system of claim 16 wherein said imaging modalities are located on a single gantry.

19. A patient imaging system comprising:
a single gantry having an MRI imaging modality and a CT imaging modality;
said MRI imaging modality and said CT imaging modality operably configured to obtain a simultaneous or concurrent signal acquisition from each imaging modality that creates a plurality of datasets directly involving said region-of-interest of the patient for performing reconstruction of said region-of-interest; and
configuring a processor to process said plurality of datasets to generate an image representation of said region-of-interest of the patient.

20. The imaging system of claim 19 wherein said MRI imaging modality has a homogeneous or inhomogeneous magnetic background.

21. The system of claim 2 wherein said gantry includes a rotational ring containing at least one x-ray tube, at least one detector targeting a region of interest, and at least one SPECT detector camera targeting said region of interest in order to collect datasets for image reconstruction over said region of interest.
